(19) United States
(12) Patent Application Publication
(10) Pub. No.: US 2020/0055848 A1
(43) Pub. Date: Feb. 20, 2020

(54) FURANONE DERIVATES AND METHODS OF USE THEREOF

(71) Applicant: CARNA BIOSCIENCES, INC., Kobe-shi, Hyogo (JP)

(72) Inventors: Takayuki IRIE, Kobe (JP); Ayako SAWA, Osaka (JP); Masaaki SAWA, Ibaraki (JP); Tokiko ASAMI, Kobe (JP); Yoko FUNAKOSHI, Tokyo (JP); Chika TANIYAMA, Tokyo (JP)

(21) Appl. No.: 16/347,153

(22) PCT Filed: Nov. 2, 2017
(86) PCT No.: PCT/JP2017/039822

§ 371 Date: May 2, 2019

Related U.S. Application Data
(60) Provisional application No. 62/417,943, filed on Nov. 4, 2016.
(60) Provisional application No. 62/447,823, filed on Jan. 18, 2017.

(51) Int. Cl. C07D 471/04; A61P 35/00

(52) U.S. Cl. CPC C07D 471/04; A61P 35/00

(57) ABSTRACT

Herein disclosed are compounds, compositions, kits, and methods of treating cancers using 7-azaindolyl furanone/thiophene derivatives. These derivatives inhibit serine-threonine kinase Cdc7, a recognized anticancer target affecting DNA replication. Further, the compounds disclosed herein possess potent inhibitory activity in the presence of adenosine triphosphate (ATP), demonstrate significant kinase selectivity, and offer advantages over known Cdc7 inhibitors with prolonged half-life and inhibitory effects.

### CROSS-REFERENCE TO RELATED APPLICATIONS

This application claims the benefit of U.S. Provisional Application No. 62/417,943, filed Nov. 4, 2016, and U.S. Provisional Application No. 62/447,823, filed Jan. 18, 2017. Each is hereby incorporated in its entirety by reference.

### BACKGROUND ART

### Field of the Invention

In general, the invention relates to compounds, compositions, kits, and methods useful for treating cancer. More specifically, the compounds and compositions disclosed herein are serine-threonine kinase inhibitors. These compounds and methods are broadly useful to treat cancers that are affected by or are associated with Cdc7 activity. Notably, the compounds disclosed herein possess potent inhibitory activity in the presence of adenosine triphosphate (ATP) and demonstrate significant kinase selectivity.

### Description of the Related Art

Cancer is a group of diseases caused by uncontrolled, unlimited growth of cells within a living body. Since cancer cells usually grow faster than normal cells, cancers are capable of being treated by controlling the replication of DNA during the cell division, particularly during the division of chromosomes.

Cdc7 is a serine-threonine protein kinase and is an enzyme which is essential for the initiation of DNA replication in the cell cycle. Specifically, Cdc7 forms a complex with cofactors such as Dbf4 (ASK), and phosphorylates its substrate, MCM (mini-chromosome maintenance) proteins. It is purported that this phosphorylation results in assembly of Cdc45 and a DNA polymerase on the DNA to form an MCM complex, thereby initiating the DNA replication.

Significant interest has arisen in Cdc7 as an anticancer target since the expression level of Cdc7 is frequently elevated in various cancer cell lines and human tumor tissues. It has been found that Cdc7 is overexpressed not only in commonly established cell lines derived from human tumors, but also in cells taken from live tissues.

Certain Cdc7 inhibitors have been demonstrated to affect the growth of human tumor cells, such as HeLa and HCT 116 cells, while exhibiting only limited effects on normal cells.

Therefore, selective inhibitors of Cdc7 are expected to have an effective therapeutic effect against various types of cancer.

Unfortunately, most of the identified Cdc7 inhibitors to date only show moderate inhibitory Cdc7 activity and weak cellular activity.

What is needed therefore is the development of serine-threonine kinase and Cdc7 inhibitors to overcome the weaknesses of other therapeutic candidates. To that end the inventors have found novel serine-threonine kinase inhibitors insensitive or resistant to high ATP concentration.

### SUMMARY OF INVENTION

Cdc7 has a relatively low Kₘ for ATP (Kₘₐₚₚ=2.8 μM) compared with other kinases, and it is postulated that this high affinity for ATP is a bottleneck to generate an effective inhibitor in vivo because ATP competitive inhibitors are competed out by the high concentration of ATP in a cell and as a result, the inhibitor loses potency. Therefore, the inventors implemented a high-throughput screening (HTS) over a compound library in the presence of 100 μM ATP to find compounds that are insensitive or resistant to high ATP concentration. Such a concentration corresponds to 36-times higher than the Kₘ value, and well correlates to the ATP concentration in a cell.

Herein the inventors have discovered novel 7-azaindolyl furanone and thiophene derivatives from structure-activity relationship studies. These particular derivatives are potent, selective Cdc7 inhibitors. Moreover, these compounds display significant potency for Cdc7 inhibition in the presence of high ATP concentration (i.e. at physiological ATP concentrations). Finally, these compounds were then submitted for cancer cell and kinetic studies.

Disclosed herein are compounds that selectively inhibited Cdc7 with slow off-rate kinetics and demonstrate a prolonged down-regulation of MCM2 phosphorylation in tumor cells.

In some aspects, the present disclosure includes methods of treating a cancer with one or more compounds or compositions disclosed herein. In one aspect, the cancer that is associated with, affected by, or that over-expresses a serine-threonine kinase. In one aspect, the cancer is a cancer that is associated with, affected by, or that over-expresses Cdc7 kinase.

In some aspects, the present disclosure includes methods of inhibiting a serine-threonine kinase, either in-vitro or in-vivo. In some aspects, the present disclosure includes methods of inhibiting Cdc7 kinase, either in-vitro or in-vivo. In some aspects, the present disclosure includes methods of selectively inhibiting Cdc7 kinase in the presence of ATP, either in-vitro or in-vivo. In some aspects, the present disclosure includes methods of selectively inhibiting Cdc7 kinase in the presence of ATP and one or more other kinases, either in-vitro or in-vivo.

In some aspects, the present disclosure includes methods of selectively inhibiting, or compounds that selective inhibit, of one of: Cdc7, CLK1, CLK2, GSK3α, GSK3β, DYRK1B, Erk1, Erk2, PIM1, or p70S6K. In some aspects, the present disclosure includes methods of selectively inhibiting, or compounds that selective inhibit, Cdc7 in the presence of at least one more enzyme selected from the group consisting of: CLK1, CLK2, GSK3α, GSK3β, DYRK1B, Erk1, Erk2, PIM1, and p70S6K.

In some aspects, the present disclosure includes methods of down-regulation of MCM2 phosphorylation using one or more one or more compounds disclosed herein.

In some aspects, the present disclosure includes a kit that includes a composition comprising one or more compounds disclosed herein and instructions for use.

In some aspects, the present disclosure includes one or more compounds disclosed herein for use in treating a cancer. In some aspects, the present disclosure includes one or more compounds disclosed herein for use in treating a cancer that is associated with, affected by, or that over-expresses a serine-threonine kinase or one that is associated with, affected by, or that over-expresses Cdc7 kinase.

In some aspects, methods are provided for alleviating or ameliorating a condition or disorder from cancer that is affected by or associated with the enzymatic activity of Cdc7.

In one aspect, pharmaceutical compositions are provided that include an effective amount of one or more compounds of formula I-IV described herein and one or more pharmaceutically acceptable excipients.

In some aspects, methods are provided for inhibiting Cdc7 that includes contacting (in vivo or in vitro) cells (including neurons/microglia/invading macrophages) with an effective amount of one or more compounds of formula I-IV described herein.

In certain embodiments, methods are provided for selectively inhibiting one of: Cdc7, CLK1, CLK2, GSK3α, GSK3β, DYRK1B, Erk1, Erk2, PIM1, or p70S6K that includes contacting (in vivo or in vitro) cells (including neurons/microglia/invading macrophages) with an effective amount of one or more compounds of formula I-IV described herein.

In some aspects, methods are provided for treating a cancer associated with, affected by, or that over-expresses a serine-threonine kinase or one that is associated with, affected by, or that over-expresses Cdc7 kinase, where the method comprises administering to a subject in need of treatment an effective amount of one or more compounds of formula I-IV, or a pharmaceutical composition comprising one or more pharmaceutically acceptable excipients and an effective amount of one or more compounds of formula I-IV described herein.

In one aspect, an article of manufacture is provided for use in inhibiting Cdc7 and treating a cancer associated with, affected by, or that over-expresses a serine-threonine kinase or one that is associated with, affected by, or that over-expresses Cdc7 kinase, wherein the article comprises one or more compounds of formula I-IV as provided herein. The cancers affected by or associated with Cdc7 are also provided herein. In some embodiments, the article of manufacture further includes a label with instructions for using the composition to treat the cancer.

These and other embodiments are described in further detail herein.

### BRIEF DESCRIPTION OF DRAWINGS

FIGS. 1A and 1B show the dose response curves of compound 13 for Cdc7 inhibition in the presence of various ATP concentrations: 5 μM (closed circle), 25 μM (open circle), 100 μM (closed triangle), 1000 μM (open triangle). The enzymatic assays were performed without (a) or with pre-incubation (b).

FIG. 2 is a graph of the results obtained from the rapid dilution assay for Cdc7 inhibitors. Recovery of enzymatic activity was monitored by formation of the phosphorylated product. DMSO control (closed circle), compound 1 (open triangle), compound 13 (open square).

FIGS. 3A and 3B illustrate the effects of Cdc7 inhibition by compound 13 in Colo-205 cells. a) Western blot analysis of extracts prepared from Colo-205 cells at 48 h after treatment with DMSO control (0 μM) or compound 13 at indicated concentrations. b) Flow chemistry analysis of Colo-205 cells treated for 48 h with DMSO control (0 μM) and the indicated concentration of compound 13. DNA content was measured by FACS after propidium iodide staining.

FIG. 4 shows the Western Blot results for the recovery of MCM2 phosphorylation at Ser53 after treatment with 13. Colo-205 cells were treated with or without compounds for 48 h. After inhibitor washout with compound-free media, the cells were lysed at the indicated time and subjected to western blot analysis.

### DESCRIPTION OF EMBODIMENTS

Compounds, compositions, kits, and methods of the present disclosure inhibit serine-threonine kinases. Specifically, the compounds, compositions, kits, and methods of the present disclosure are useful for the therapy or treatment of cancers and specifically, those cancers that are associated with, affected by, or that over-express Cdc7 kinase. Even more specifically, compounds, compositions, kits, and methods disclosed herein are effective therapy or treatment for cancers where selective inhibition of Cdc7 in the presence of physiological levels of ATP is required.

### Definitions

Terms used in the claims and specification are defined as set forth below unless otherwise specified.

As used herein and in the appended claims, singular articles such as “a,” “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

The term “ameliorating” refers to any therapeutically beneficial result in the treatment of a disease state, e.g., an inflammatory disease state, including lessening in the severity or progression thereof.

The term “in vitro” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term “therapeutically effective amount” is an amount that is effective to ameliorate a symptom of a disease.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology can exist as solvates, especially hydrates. Hydrates may form during manufacture of the compounds or compositions comprising the compounds, or hydrates may form over time due to the hygroscopic nature of the compounds. Compounds of the present technology can exist as organic solvates as well, including DMF, ether, and alcohol solvates among others. The identification and preparation of any particular solvate is within the skill of the ordinary artisan of synthetic organic or medicinal chemistry.

“Subject” refers to a mammalian organism treated using a compound of the present invention. The “subject” can be a human or non-human mammalian organism.

“Tautomer” refer to alternate forms of a compound that differ in the position of a proton, such as enol-keto and imine-enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

“Treating” or “treatment” of a disease or disorder in a subject refers to 1) preventing the disease or disorder from occurring in a subject that is predisposed or does not yet display symptoms of the disease or disorder; 2) inhibiting the disease or disorder or arresting its development; or 3) ameliorating or alleviating the cause of the regression of the disease or disorder.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

As used herein, the following definitions shall apply unless otherwise indicated. Further, if any term or symbol used herein is not defined as set forth below, it shall have its ordinary meaning in the art.

As used herein, the phrase “modulating or inhibiting (the activity of)” refers to use of any agent capable of altering the cellular expression levels and/or biological activity of the protein or enzyme. In some embodiments, an agent that modulates or inhibits the biological activity of the protein or enzyme directly interferes with the expression (such as transcription, splicing, transport, etc.) of the gene encoding the of the protein or enzyme. In other embodiments, an agent that modulates or inhibits the activity of the protein or enzyme directly interferes with the biological activity or production of the of the protein or enzyme (such as though inhibition of translation, post-translational modifications, intracellular transport, disruption of interactions between one or more proteins, etc.). In yet other embodiments, an agent that modulates or inhibits the activity of the protein or enzyme does not directly affect the expression level or activity of the protein or enzyme but, instead, alters the activity or expression levels of a different protein whose function directly impacts the expression or activity of the protein or enzyme (such as, for example, Dbf4).

As used herein, the term “inhibit,” “decrease” and grammatical derivations thereof, refers to the ability of an agent to block, partially block, interfere, reduce or deactivate a pathway or mechanism of action. Thus, one of ordinary skill in the art would appreciate that the term “inhibit” or “decrease” encompasses a complete and/or partial loss of activity, e.g., a loss in activity by at least 10%, in some embodiments, a loss in activity by at least 20%, 30%, 50%, 75%, 95%, 98%, and up to and including 100%.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent can be administered prophylactically to prevent the onset of a disease, disorder, or condition, or to prevent the recurrence of a disease, disorder, or condition.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some aspects, ±100% in some aspects ±50%, in some aspects ±20%, in some aspects ±10%, in some aspects ±5%, in some aspects ±1%, in some aspects ±0.5%, and in some aspects ±0.1% from the specified amount, as such variations are appropriate to perform the disclosed methods or employ the disclosed compositions.

Unless defined otherwise, all technical and scientific terms used herein have the meaning commonly understood by a person skilled in the art to which this invention belongs.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃ CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkenyl” refers to straight or branched hydrocarbyl groups having from 1 to 6 carbon atoms and preferably 2 to 4 carbon atoms and having at least 1 and preferably from 1 to 2 sites of unsaturation (>C═C<). Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH). Cₓ alkynyl refers to an alkynyl group having x number of carbon atoms.

“Substituted alkyl” refers to an alkyl group having from 1 to 5, preferably 1 to 3, or more preferably 1 to 2 substituents selected from the group consisting of alkoxy, substituted alkoxy, acyl, acylamino, aminocarbonylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

In some embodiments the substituted alkyl groups include halogenated alkyl groups and particularly halogenated methyl groups such as trifluoromethyl, difluromethyl, fluoromethyl and the like.

“Cycloalkyl” or “Cyclyl alkyl” refers to a saturated or partially saturated, but not aromatic, group having from 1 to 10 ring carbon atoms and no heteroatoms. Cycloalkyl encompasses single ring or multiple condensed rings, including fused bridged and spiro ring systems. In fused ring systems, one or more of the rings can be cycloalkyl, aryl, heterocycloalkyl, or heteroaryl provided that the point of attachment is through the original non-aromatic cycloalkyl ring.

“Substituted alkenyl” refers to alkenyl groups having from 1 to 3 substituents, and preferably 1 to 2 substituents, selected from the group consisting of alkoxy, substituted alkoxy, acyl, acylamino, aminocarbonylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, aminocarbonylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxyl or thiol substitution is not attached to an acetylenic carbon atom.

“Ar” refers to any group which is aromatic. This group must be cyclic; however, it may contain heteroatoms or may not.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein. Preferred substituted alkyl groups in —O-(substituted alkyl) include halogenated alkyl groups and particularly halogenated methyl groups such as trifluoromethyl, difluromethyl, fluoromethyl and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups —NR³⁰C(O)alkyl, —NR³⁰C(O)substituted alkyl, —NR³⁰ C(O)cycloalkyl, —NR³⁰C(O)substituted cycloalkyl, —NR³⁰C(O)alkenyl, —NR³⁰ C(O)substituted alkenyl, alkoxy, substituted alkoxy-NR³⁰C(O)alkynyl, —NR³⁰ C(O)substituted alkynyl, —NR³⁰C(O)aryl, —NR³⁰C(O)substituted aryl, —NR³⁰ C(O)heteroaryl, —NR³⁰C(O)substituted heteroaryl, —NR³⁰C(O)heterocyclic, and —NR³⁰ C(O)substituted heterocyclic wherein R³⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminoacyl” refers to the groups H—C(N)—, alkyl-C(N)—, substituted alkyl-C(N)—, alkenyl-C(N)—, substituted alkenyl-C(N)—, alkynyl-C(N)—, substituted alkynyl-C(N)—, cycloalkyl-C(N)—, substituted cycloalkyl-C(N)—, aryl-C(N)—, substituted aryl-C(N)—, heteroaryl-C(N)—, substituted heteroaryl-C(N)—, heterocyclic-C(N)—, and substituted heterocyclic-C(N)—, wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein. Acyl includes the “acetyl” group CH₃C(N)—.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³¹R³² where R³¹ and R³² are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, substituted heterocyclic, and substituted sulfonyl and wherein R³¹ and R³² are optionally joined, together with the nitrogen bound thereto to form a heterocyclic or substituted heterocyclic group, provided that R³¹ and R³² are both not hydrogen, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic, and substituted heterocyclic are as defined herein. When R³¹ is hydrogen and R³² is alkyl, the substituted amino group is sometimes referred to herein as alkylamino. When R³¹ and R³² are alkyl, the substituted amino group is sometimes referred to herein as dialkylamino. When referring to a monosubstituted amino, it is meant that either R³¹ or R³² is hydrogen but not both. When referring to a disubstituted amino, it is meant that neither R³¹ nor R³² are hydrogen.

“Aminocarbonyl” refers to the group —C(O)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminoacyl carbonyloxy” refers to the group —C(NR³³)OR³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonyl” refers to the group —C(S)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the group —NR³⁰C(O)NR³³R³⁴ where R³⁰ is hydrogen or alkyl and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the group —NR³⁰C(S)NR³³R³⁴ where R³⁰ is hydrogen or alkyl and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the group —O—C(O)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyl” refers to the group —SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyloxy” refers to the group —O—SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonylamino” refers to the group —NR³⁰—SO₂NR³³R³⁴ where R³⁰ is hydrogen or alkyl and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Amidino” refers to the group —C(═NR³⁵)NR³³R³⁴ where R³³, R³⁴, and R³⁵ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, aminocarbonylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR³⁰—C(O)O-alkyl, —NR³⁰—C(O)O-substituted alkyl, —NR³⁰—C(O)O-alkenyl, —NR³⁰—C(O)O-substituted alkenyl, —NR ³⁰—C(O)O-alkynyl, —NR³⁰—C(O)O-substituted alkynyl, —NR³⁰—C(O)O-aryl, —NR³⁰—C(O)O-substituted aryl, —NR³⁰—C(O)O-cycloalkyl, —NR³⁰—C(O)O-substituted cycloalkyl, —NR³⁰—C(O)O-heteroaryl, —NR³⁰—C(O)O-substituted heteroaryl, —NR³⁰—C(O)O-heterocyclic, and —NR³⁰—C(O)O-substituted heterocyclic wherein R³⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” refers to the group —C≡N.

“Cycloalkyl” refers to a saturated or unsaturated but nonaromatic cyclic alkyl groups of from 3 to 10 carbon atoms having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring.

“Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, aminocarbonylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Ethylene glycol” refers to the group —O—CH₂CH₂—O-E, wherein E is either H or CH₃.

“Guanidino” refers to the group —NHC(═NH)NH₂.

“Substituted guanidino” refers to —NR³⁶C(═NR³⁶)N(R³⁶)₂ where each R³⁶ is independently selected from the group consisting of hydrogen, alkyl, substituted alkyl, aryl, substituted aryl, heteroaryl, substituted heteroaryl, heterocyclic, and substituted heterocyclic and two R³⁶ groups attached to a common guanidino nitrogen atom are optionally joined together with the nitrogen bound thereto to form a heterocyclic or substituted heterocyclic group, provided that at least one R³⁶ is not hydrogen, and wherein said substituents are as defined herein.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Cₓ cycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryl include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothI zole, phenazine, isoxazole, phenoxazine, phenothI zine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiomorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O).

“Phthalimido” refers to the group

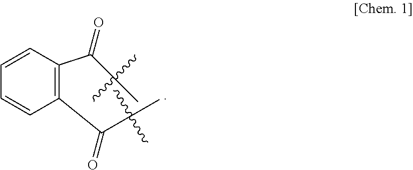 [SMILES: *C(=O)c1ccccc1C(*)=O]

Phthalimide functional groups are well known in the art and can be generated by covalently bonding a nitrogen atom to a C₆H₄(CO)₂ group.

“Polyethylene glycol” refers to the group —O—(CH₂CH₂—O)ₙ-E, wherein E is either H or CH₃, where n is between 2-20,000.

“Spirocyclic ring system” refers to a ring system with two rings that has a single ring carbon atom in common to both rings. Herein used the term bicyclic can incorporate up to four heteroatoms in either ring.

“Bicyclic ring system” refers to a ring system with two rings that has two ring carbon atoms in common, and which can located at any position along either ring. Herein used the term bicyclic ring system can incorporate up to four heteroatoms in either ring.

“Sulfinyl” refers to the divalent group —SO—.

“Sulfonyl” refers to the divalent group —S(O)₂—.

“Substituted sulfonyl” refers to the group —SO₂-alkyl, —SO₂-substituted alkyl, —SO₂—OH, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—. Preferred substituted alkyl groups on the substituted alkyl-SO₂— include halogenated alkyl groups and particularly halogenated methyl groups such as trifluoromethyl, difluromethyl, fluoromethyl and the like.

“Substituted sulfinyl” refers to the group —SO-alkyl, —SO-substituted alkyl, —SO-alkenyl, —SO-substituted alkenyl, —SO-cycloalkyl, —SO-substituted cycloalkyl, —SO-aryl, —SO-substituted aryl, —SO-heteroaryl, —SO-substituted heteroaryl, —SO-heterocyclic, —SO-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfinyl includes groups such as methyl-SO—, phenyl-SO—, and 4-methylphenyl-SO—. Preferred substituted alkyl groups on the substituted alkyl-SO— include halogenated alkyl groups and particularly halogenated methyl groups such as trifluoromethyl, difluromethyl, fluoromethyl and the like.

“Sulfonyloxy” or “substituted sulfonyloxy” refers to the group —OSO₂-alkyl, —OSO₂-substituted alkyl, —OSO₂—OH, —OSO₂-alkenyl, —OSO₂-substituted alkenyl, —OSO₂-cycloalkyl, —OSO₂-substituted cycloalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Substitution” or “substitution” generally refers groups which are covalently bonded to an atom to replace a hydrogen atom. The atom in this general context can be a carbon atom or a heteroatom, for example a nitrogen atom.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Mercapto” or “thiol” refers to the group —SH.

“Formyl” refers to the group —C(O)H.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein. Preferred substituted alkyl groups on —S-(substituted alkyl) include halogenated alkyl groups and particularly halogenated methyl groups such as trifluoromethyl, difluromethyl, fluoromethyl and the like.

Herein the term “-position” refers to the spot on which the substituting group is placed on the a cycle to which it is bonded, according to the IUPAC numbering and nomenclature system for organic compounds. For example, if the cycle from which an R group is substituting is

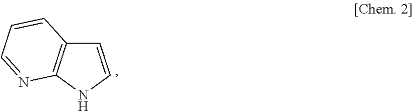

then the number according to IUPAC rules would be

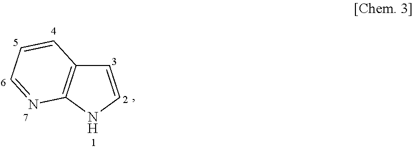

So then using this example, the “5-position” for any R group would be represented by

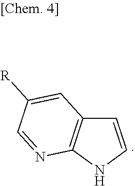

The same numbering shall proceed with IUPAC rules for all cyclic groups including those without heteroatoms.

Herein the term “exocyclic” refers to any bond or group that is attached to the outside of a cyclic group.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and d or 1 enriched stereomeric mixtures, as well as the individual optical isomers can be isolated or synthesized so as to be substantially free of their enantiomeric or diastereomeric partners, and these stereoisomers are all within the scope of the present technology.

Herein any substituted functional group is substituted at from one to three different positions, and those one to three substituting groups are capable of each independently being substituted at one to three positions, wherein any and each substituting group is independently selected from the group consisting of: halogen, hydroxyl, C₁-C₈ alkyl, substituted C₁-C₈ alkyl, C₁-C₈ alkenyl, substituted C₁-C₈ alkenyl, C₁-C₈ alkynyl, substituted C₁-C₈ alkynyl, acyl, acylamino, aminocarbonylamino, aminoacyl, acyloxy, amino, substituted amino, aminocarbonyl, aminothiocarbonyl, aminoacyl carbonyloxy, aminocarbonylamino, aminothiocarbonylamino, aminocarbonyloxy, aminosulfonyl, aminosulfonyloxy, aminosulfonylamino, amidino, C₁-C₈ alkoxy, substituted C₁-C₈ alkoxy, C₃-C₇ aryl, substituted C₃-C₇ aryl, C₃-C₇ aryloxy, substituted C₃-C₇ aryloxy, C₃-C₇ arylthio, substituted C₃-C₇ arylthio, carboxyl, carboxyl ester, (carboxyl ester)amino, (carboxyl ester)oxy, C₃-C₁₀ cycloalkyl, substituted C₃-C₁₀ cycloalkyl, C₃-C₇ heterocycloalkyl, guanidino, substituted guanidino, C₃-C₇ heteroaryloxy, C₃-C₇ substituted heteroaryloxy, C₃-C₇ heteroarylthio, C₃-C₇ substituted heteroarylthio, sulfonyl, substituted sulfonyl, sulfinyl, substituted sulfinyl, sulfonyloxy, substituted sulfonyloxy, thioacyl, alkylthio, substituted alkylthio, C₃-C₇ heteroaryl, and substituted C₃-C₇ heteroaryl.

Herein any and all heteroaryl and heterocycloalkyl substituents may contain up to four heteroatoms selected from the group consisting of: O, N, and S.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that each functional group is substituted (at from one to three positions) and that any and all of those substituent groups may be substituted one more time (at from one to three positions).

It is understood that the above definitions are not intended to include impermissible substitution patterns (e.g., methyl substituted with 5 fluoro groups). Such impermissible substitution patterns are well known to the skilled artisan.

Throughout this application, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

Compounds

Disclosed herein are compounds, compositions, and methods of using said compounds or compositions to inhibit serine-threonine kinases or Cdc7. Further compounds of the present invention selectively inhibit serine-threonine kinases in the presence of ATP and/or other kinases.

Compounds of formula I-IV selectively inhibit Cdc7 in the presence of ATP and/or other kinases. Such compounds are useful for the treatment or therapy of cancers and other diseases or disorders that are associated with, affected by, or that over-express Cdc7.

In one aspect, the present disclosure provides two or more compounds of Formula I-IV described herein.

In one aspect, a compound of the present disclosure is according to formula (I):

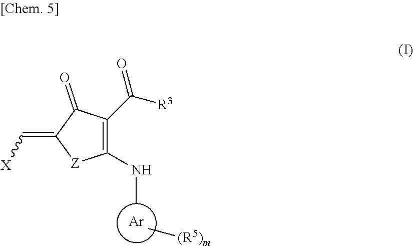

wherein X is

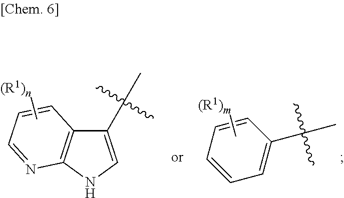

### Z is O or S;

n is from 1-3;
m is from 0-4;
each R¹ and R⁵ are independently selected from the group consisting of: halogen, hydrogen, hydroxy, C₁-C₈ alkyl, substituted C₁-C₈ alkyl, C₁-C₈ alkoxy, and substituted C₁-C₈ alkoxy;
R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded through a nitrogen atom to the carboxy group to form

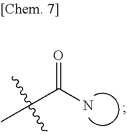 [SMILES: *C(N)=O]

Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
or a tautomer and/or a pharmaceutically acceptable salt thereof.

In one aspect, a compound of the present disclosure is according to formula I,

wherein Z is O, X is

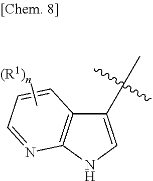

and n is 3 and each R¹ is F.

In one aspect, a compound of the present disclosure is according to formula I, wherein Z is O, X is

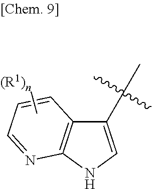

and n is 2 and each R¹ is Cl.

In one aspect, a compound of the present disclosure is according to formula I,

wherein X is

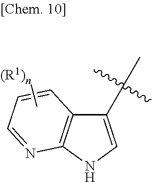

and n is 2 and each R¹ is F.

In one aspect, a compound of the present disclosure is according to formula I, wherein X is

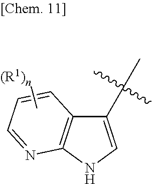

and m is 2, n is 2 and each R¹ is OMe.

In one aspect, a compound of the present disclosure is according to formula I, wherein X is

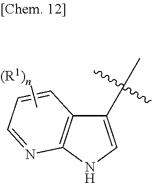

and m is 2, n is 2 and each R¹ is F.

In one aspect, a compound of the present disclosure is according to formula I,

wherein X is

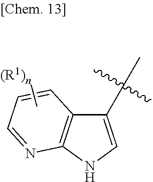

and m is 2, n is 2, both R¹ groups join to form a fused bicyclic ring according to

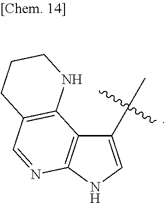 [SMILES: *c1c[nH]c2ncc3c(c12)NCCC3]

In one aspect, a compound of the present disclosure is according to formula I wherein X is

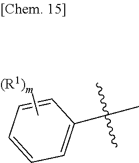

and m is 5 and each R¹ is F.

In one aspect, a compound of the present disclosure is according to formula I wherein Z is O, X is

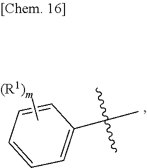

m is 2, one R¹ is OH and one R¹ is F.

In one aspect, a compound of the present disclosure is according to formula I wherein Z is S, X is

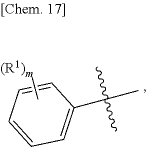

m is 2, one R¹ is F and one R¹ is F.

In one aspect, a compound of the present disclosure is according to formula I wherein Z is S, X is

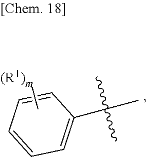

m is 2, both R¹ groups join to form a fused bicyclic ring according to

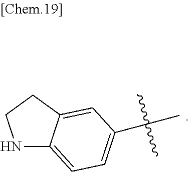 [SMILES: *c1ccc2c(c1)CCN2]

In one aspect, a compound of the present disclosure is according to formula (II):

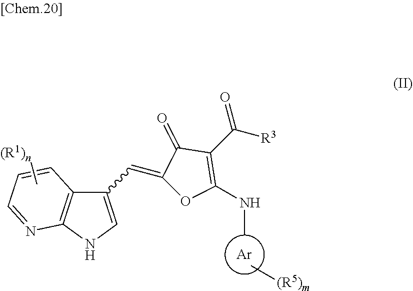

wherein

n is from 2-3;

m is from 0-4;

each R¹ and R⁵ are independently selected from the group consisting of: halogen, hydrogen, hydroxy, C₁-C₈ alkyl, substituted C₁-C₈ alkyl, C₁-C₈ alkoxy, and substituted C₁-C₈ alkoxy;

R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded thru a nitrogen atom to the carboxy group to form

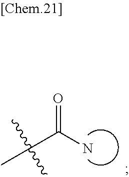 [SMILES: *C(N)=O]

Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
provided that at least two of the R¹ are not hydrogen;
or a tautomer and/or a pharmaceutically acceptable salt thereof.

In one aspect, a compound of the present disclosure is the compound of formula II, wherein Ar is

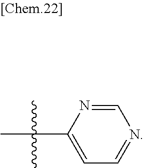 [SMILES: *c1ccncn1]

In one aspect, a compound of the present disclosure is the compound of formula II, wherein n is 2, m is 4, and Ar is Ph.

In one aspect, a compound of the present disclosure is the compound of formula II, wherein n is 3, m is 1, one R¹ is F substituted at the 4-position, one R¹ is F substituted at the 5-position, one R¹ is F substituted at the 6-position, R³ is

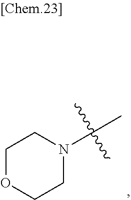 [SMILES: *N1CCOCC1]

### Ar is

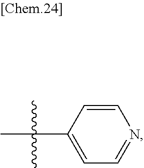 [SMILES: *c1ccncc1]

and R⁵ is Cl substituted at the 2-position.

In one aspect, a compound of the present disclosure is the compound of formula II, wherein n is 2, m is 2, R¹ is F substituted at the 5-position, R³ is

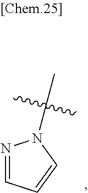 [SMILES: *n1cccn1]

Ar is Ph, one R⁵ is Cl substituted at the 2-position and one R⁵ is Cl substituted at the 4-position.

In one aspect, a compound of the present disclosure is the compound of formula II, wherein n is 2, m is 2, one R¹ is F substituted at the 4-position, one R¹ is F substituted at the 5-position, R³ is OMe, Ar is Ph, one R⁵ is OMe substituted at the 2-position and one R⁵ is OMe substituted at the 4-position.

In one aspect, a compound of the present disclosure is the compound of claim 2, wherein n is 3, m is 1, one R¹ is F substituted at the 4-position, one R¹ is F substituted at the 5-position, one R¹ is F substituted at the 6-position, R³ is OEt, Ar is Ph, and R⁵ is F substituted at the 2-position.

In one aspect, a compound of the present disclosure is the compound of formula II, wherein n is 2, m is 0, one R¹ is OMe substituted at the 4-position, one R¹ is OMe substituted at the 5-position, R³ is hydroxy, and Ar is

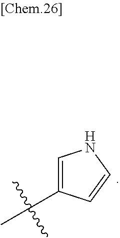 [SMILES: *c1cc[nH]c1]

In one aspect, a compound of the present disclosure is the compound of formula II, wherein n is 3, m is 1, one R¹ is Me substituted at the 4-position, one R¹ is Me substituted at the 5-position, one R¹ is Me substituted at the 6-position, R³ is hydroxy, Ar is Ph, and R⁵ is Cl substituted at the 2-position.

In one aspect, a compound of the present disclosure is the compound of formula II, wherein n is 2, m is 0, one R¹ is F substituted at the 4-position, one R¹ is F substituted at the 5-position, R³ is

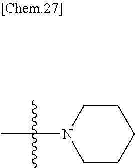 [SMILES: *N1CCCCC1]

### and Ar is

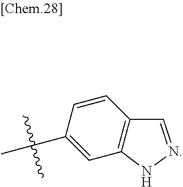 [SMILES: *c1ccc2cn[nH]c2c1]

In one aspect, a compound of the present disclosure is the compound of formula II, wherein n is 2, R³ is

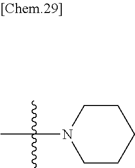 [SMILES: *N1CCCCC1]

### and Ar is

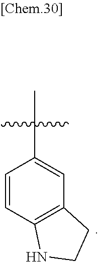 [SMILES: *c1ccc2c(c1)CCN2]

In one aspect, a compound of the present disclosure is the compound of formula II, wherein n is 2, R³ is

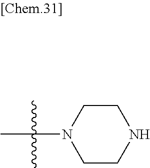 [SMILES: *N1CCNCC1]

### and Ar is

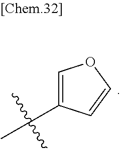 [SMILES: *c1ccoc1]

In one aspect, a compound of the present disclosure is the compound of formula II, wherein n is 2, R³ is

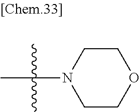 [SMILES: *N1CCOCC1]

### and Ar is

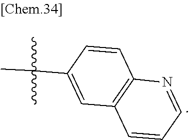 [SMILES: *c1ccc2ncccc2c1]

In one aspect, a compound of the present disclosure is the compound of formula I-IV, wherein the geometry of the exocyclic double bond is the Z-isomer.

In one aspect, a compound of the present disclosure is any compound disclosed herein, where the geometry of the exocyclic double bond is the Z-isomer.

In one aspect, a compound of the present disclosure is according to formula II wherein n is 2 and m is 1.

In one aspect, a compound of the present disclosure is according to formula II wherein n is 2 and m is 2.

In one aspect, a compound of the present disclosure is according to formula II wherein n is 2 and m is 3.

In one aspect, a compound of the present disclosure is according to formula II wherein n is 2 and m is 4.

In one aspect, a compound of the present disclosure is according to formula II wherein n is 2 and m is 2.

In one aspect, a compound of the present disclosure is according to formula II wherein n is 2 and m is 3.

In one aspect, a compound of the present disclosure is according to formula II wherein n is 2 and m is 4.

In one aspect, a compound of the present disclosure is according to formula (III):

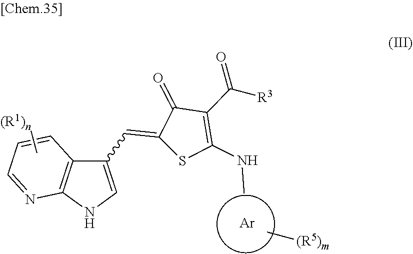

wherein
n is from 1-3;
m is from 0-4;
each R¹ and R⁵ are independently selected from the group consisting of: halogen, hydrogen, hydroxy, C₁-C₈ alkyl, substituted C₁-C₈ alkyl, C₁-C₈ alkoxy, and substituted C₁-C₈ alkoxy;
R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded thru a nitrogen atom to the carboxy group to form

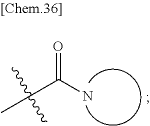 [SMILES: *C(N)=O]

Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
or a tautomer and/or a pharmaceutically acceptable salt thereof.

In one aspect, a compound of the present disclosure is according to formula III wherein n is 1, m is 1, R³ is OEt, Ar is Ph, and R⁵ is OH substituted at the 2-position. In one aspect, a compound of the present disclosure is according to formula III wherein n is 1, m is 2, R¹ is OH substituted at the 6-position, R³ is OMe, Ar is 2-pyridinyl, one R⁵ is F substituted at the 4-position and one R⁵ is Cl substituted at the 5-position. In one aspect, a compound of the present disclosure is according to formula III wherein n is 3, m is 1, each R¹ is F, R³ is OEt, Ar is Ph, and R⁵ is OH substituted at the 2-position.

In one aspect, a compound of the present disclosure is according to formula (I):

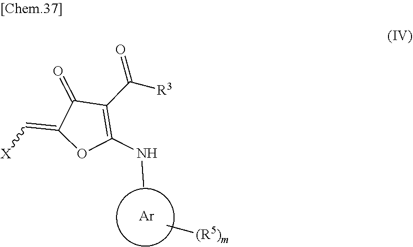

wherein

### X is

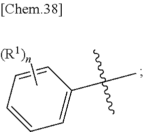

n is from 0-4;
m is from 0-4;
each R¹ and R⁵ are independently selected from the group consisting of: halogen, hydrogen, hydroxy, C₁-C₈ alkyl, substituted C₁-C₈ alkyl, C₁-C₈ alkoxy, and substituted C₁-C₈ alkoxy;
R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded through a nitrogen atom to the carboxy group to form

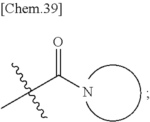 [SMILES: *C(N)=O]

Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
or a tautomer and/or a pharmaceutically acceptable salt thereof.

In one aspect, a compound of the present disclosure is according to formula IV wherein n is 0, m is 1, Ar is Ph, and R⁵ is OH substituted at the 3-position.

In one aspect, a compound of the present disclosure is according to formula IV wherein n is 1, m is 1, R¹ is OH substituted at the 2-position, Ar is Ph, and R⁵ is Ph substituted at the 4-position.

In one aspect, a compound of the present disclosure is according to formula IV wherein n is 3, m is 3, one R¹ is F substituted at the 2-position, one R¹ is CF₃ substituted at the 4-position, one R¹ is OMe substituted at the 6-position, R³ is —N-piperidine, one R⁵ is Cl substituted at the 2-position, one R⁵ is Cl substituted at the 3-position, and one R⁵ is OMe substituted at the 4-position.

In one aspect, the present disclosure provides for a kit including a composition comprising one or more compounds of formula I-IV and instructions for use.

In one aspect, the present disclosure provides for a kit including a composition comprising one or more compounds disclosed herein and instructions for use.

In one aspect, the compound is selected from the group consisting of:

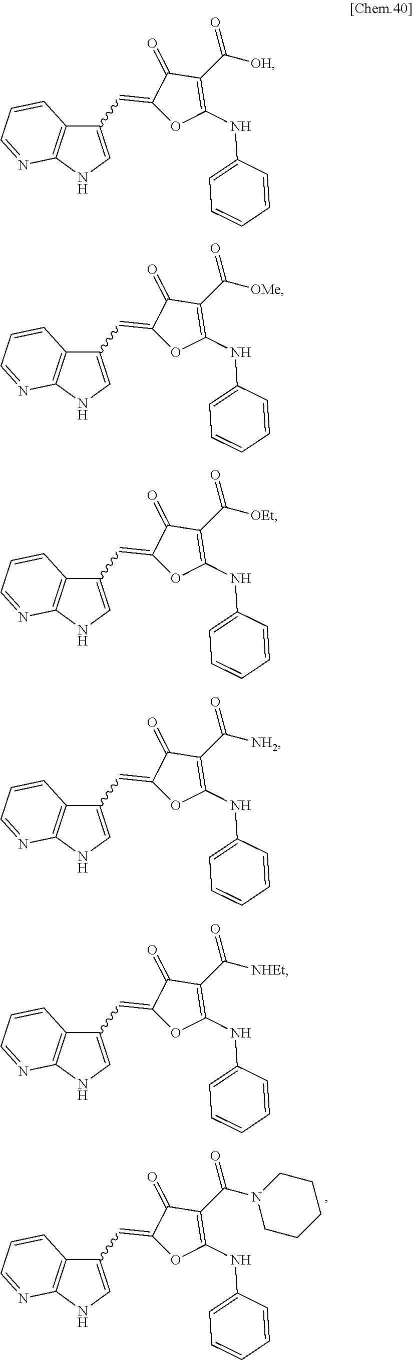

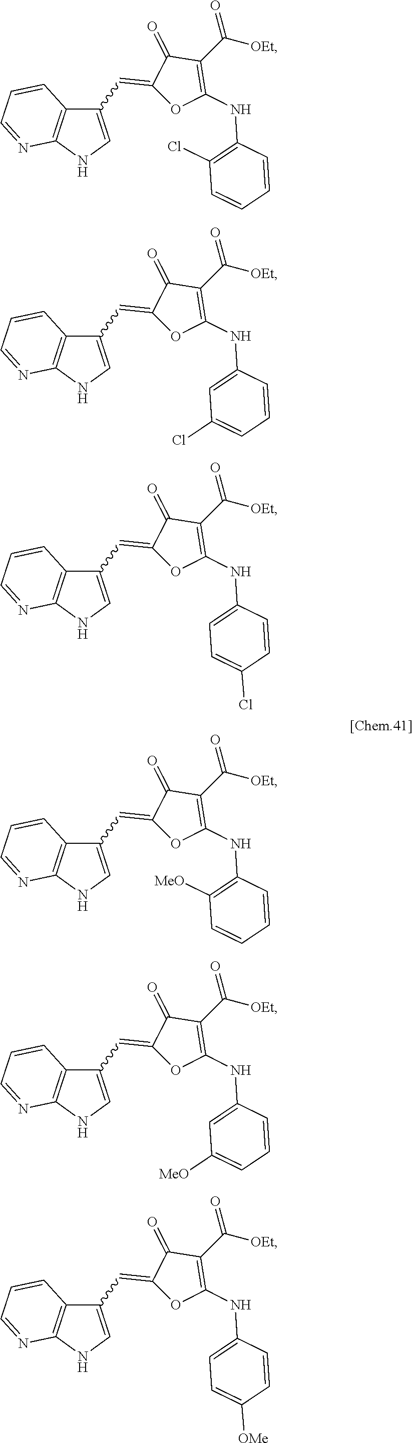

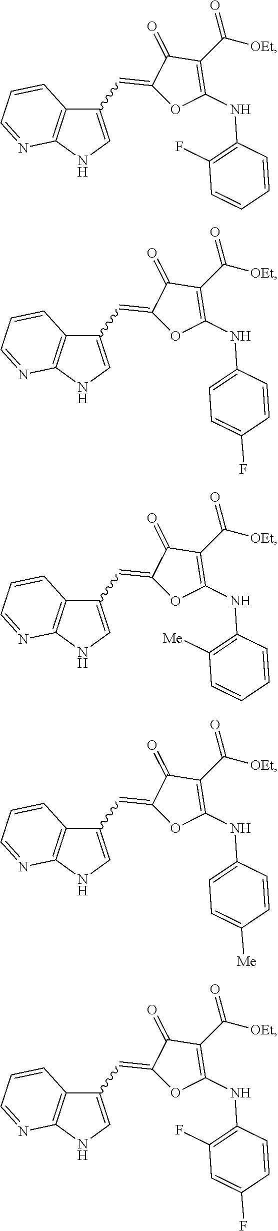

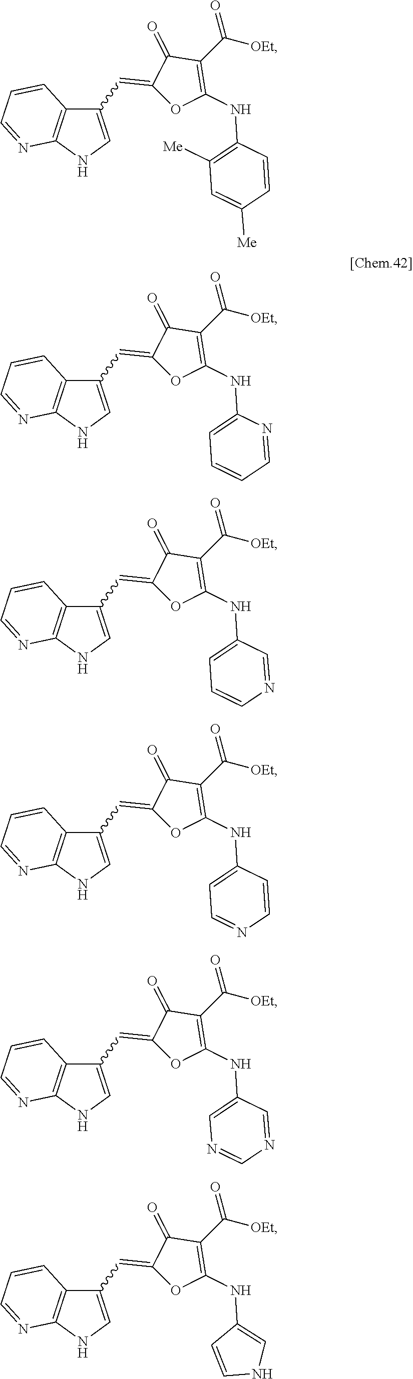

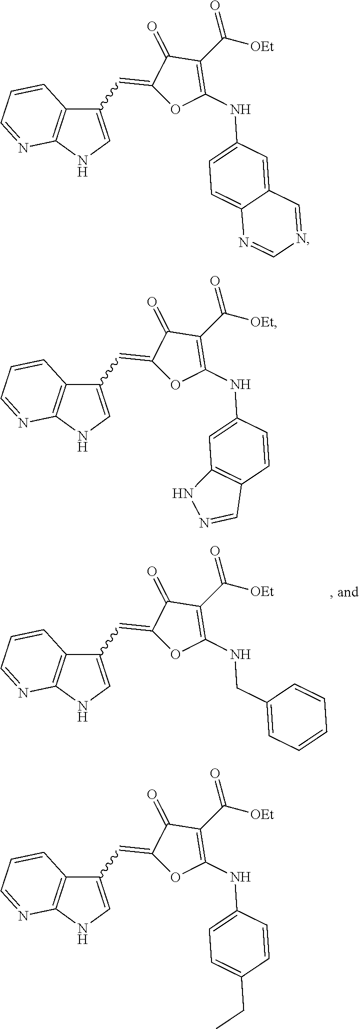

### Synthesis Routes to Compounds of the Present Technology

In a general aspect, methods to make compounds of the present disclosure involves enolate chemistry. Briefly, the requisite aromatic aldehyde is reacted with a 3(2H) furanone/thiophene derivative under basic conditions. The skilled artisan will recognize that substituents on the aldehyde and the 3(2H) furanone/thiophene may need to be appropriately protected as they may compete for the initial deprotonation of the a 3(2H) furanone/thiophene derivative or may compete as a nucleophile in the addition reaction to the aldehyde. In either event, the reaction can be slowed or even stopped. It is also evident that any substituents not be overly acidic (stop the deprotonation step) or electrophilic (compete as a side reaction with the addition reaction). The base need not be an organic base such as NaNH₂, LDA or LTMP (chelating base), rather the skilled artisan can adjust the solvent system, which only needs to be organic and aprotic (preferable ethereal), and use an inorganic base such as K₂CO₃. In other circumstances, the base can be a stronger inorganic base such as CeCO₃.This synthesis route is generally shown in Representative Synthesis 1.

Representative Synthesis 1

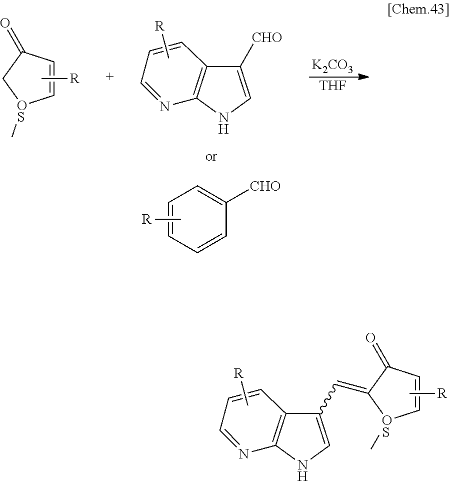

In the case of the 7-azaindolyly system, the requisite aldehyde can be prepared by reacting the appropriate azaindolyl derivative with POCl₃ in DMF in a Vilsmeier-Haack reaction. Alternatively, the appropriate azaindolyl derivative in glacial acetic acid powdered hexamethylenetetramine affords the 3-aldehyde. In the case of the phenyl system, the requisite aldehyde can be prepared facile by reducing the corresponding benzoic acid with a reducing agent. Such selective reducing agents are known in the art such as DIBAL or NaBH₃CN and the like. Finally, if the appropriately substituted phenyl or 7-azaindole compounds are not commercially available, the skilled artisan can use nucleophilic substitution and electrophilic substitution reactions on the azaindole and benzene starting materials to get the desired substituent pattern and identity.

In the case of the requisite 5-amino-3(2H)furanone/thiophene, these derivatives can be prepared by treating the aryl iso(thio)cyanate with 4-chloroacetoester.

Representative Synthesis 2

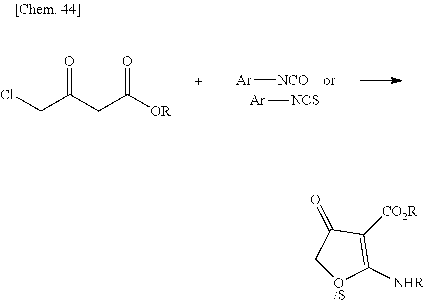

In another general aspect, the compounds of the present disclosure are alternatively made cyclization of γ-hydroxyalkynones. Briefly, a catalyst of (p-CF₃C₆H₄)₃PAuCl and AgOTf reacts with the requisite γ-hydroxyalkynones in toluene at room temperature to give substituted 3(2H)-furanones. Of course, this method may have to be modified with protecting groups as necessary with the incorporation of such strong Lewis acidic catalysts.

It is noted that the R¹-R⁵ variables for the compound structure(s) in the present disclosure are encompassed within with the R variables in these syntheses. The skilled artisan will be able to recognize each R variable and the X variables in order to translate this method to any such synthetic scheme they wish to perform to make the 7-azaindole or phenyl derivative compounds.

In one aspect, the compositions and/or compounds of the present invention or a pharmaceutically acceptable salt thereof, or a prodrug thereof, can be administered in combination with any other pharmaceutical compound approved for treating cancers that are associated with, affected by, or that over-expresses a serine-threonine kinase and/or Cdc7.

Pharmaceutical Compositions

The present technology provides novel compounds possessing serine-threonine kinase and/or Cdc7 inhibitory activity. These compounds are useful in treating a cancer that is associated with, affected by, or that over-expresses a serine-threonine kinase or a cancer that is associated with, affected by, or that over-expresses Cdc7.

In one aspect, the present disclosure provides for pharmaceutical compositions comprising one or more compounds of formula I-IV and one or more pharmaceutically acceptable excipients. In another aspect, the present disclosure provides methods for treating a cancer, cancer that is associated with, affected by, or that over-expresses a serine-threonine kinase and/or Cdc7, with an effective amount of a pharmaceutical composition comprising one or more compounds of formula I-IV and one or more pharmaceutically acceptable excipients as provided herein.

Said methods of the invention include administering a therapeutically effective amount of one or more compounds of formula I-IV disclosed herein. The compounds and solvates of the invention can be formulated in pharmaceutical compositions. These compositions can comprise a pharmaceutically acceptable excipient, carrier, buffer, stabilizer or other materials well known to those skilled in the art. The precise nature of the carrier or other material can depend on the route of administration, e.g. oral, intravenous, cutaneous or subcutaneous, nasal, intramuscular, intraperitoneal routes and the like.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

A composition can be administered alone or in combination with other treatments, either simultaneously or sequentially dependent upon the condition to be treated.

In general, the compounds of the present technology will be administered in a therapeutically effective amount by any of the accepted modes of administration for agents that serve similar utilities. The actual amount of the compound of the present technology, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, preferably once or twice a day.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences, 18th ed., Mack Publishing Co.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the present technology, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is therapeutic index, which can be expressed as the ratio LD₅₀/ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED₅₀ with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present technology is not limited to any particular composition or pharmaceutical carrier, as such may vary. In general, compounds of the present technology will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the present technology is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes therapeutic agents (which are formulated in a liquid form) to spray as a mist that is carried into the subject's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the subject's inspiratory air-stream during breathing by the device. In order to achieve a free flowing powder, therapeutic agent is formulated with an excipient such as lactose. A measured amount of therapeutic agent is stored in a capsule form and is dispensed with each actuation.

Pharmaceutical dosage forms of a compound of the present technology may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present technology can include one or more physiologically acceptable inactive ingredients that facilitate processing of active molecules into preparations for pharmaceutical use.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288, which is hereby incorporated in its entirety by reference, describes a pharmaceutical formulation having particles in the size range from 10 to 1,000 nm in which the active material is supported on a crosslinked matrix of macromolecules. U.S. Pat. No. 5,145,684, which is hereby incorporated in its entirety by reference, describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present technology in combination with at least one pharmaceutically acceptable excipient. Acceptable excipients are non-toxic, aid administration, and do not adversely affect therapeutic benefit of the claimed compounds. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the present technology in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990), which is hereby incorporated in its entirety by reference.

The present compositions may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compositions comprising a compound of the present technology formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt % of a compound of the present technology based on the total formulation, with the balance being one or more suitable pharmaceutical excipients. Preferably, the compound is present at a level of about 1-80 wt %. Representative pharmaceutical formulations are described below.

### FORMULATION EXAMPLES

The following are representative pharmaceutical formulations containing a compound of formula I-IV.

### Formulation Example 1

Tablet formulation. The following ingredients are mixed intimately and pressed into single scored tablets.

TABLE 1

| | Quantity per |
|---|---|
| Ingredient | tablet, mg |
| compound of this the present technology | 400 |
| cornstarch | 50 |
| croscarmellose sodium | 25 |
| lactose | 120 |
| magnesium stearate | 5 |

### Formulation Example 2

Capsule formulation. The following ingredients are mixed intimately and loaded into a hard-shell gelatin capsule.

TABLE 2

| | Quantity per |
|---|---|
| Ingredient | capsule, mg |
| compound of this the present technology | 200 |
| lactose, spray-dried | 148 |
| magnesium stearate | 2 |

### Formulation Example 3

Suspension formulation. The following ingredients are mixed to form a suspension for oral administration.

TABLE 3

| Ingredient | Amount | |
|---|---|---|
| compound of this the present technology | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. to 100 | mL |

### Formulation Example 4

Injectable formulation. The following ingredients are mixed to form an injectable formulation.

TABLE 4

| Ingredient | Amount |
|---|---|
| compound of this the present technology | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5

Suppository Formulation. A suppository of total weight 2.5 g is prepared by mixing the compound of the present technology with Witepsol^{(Registered Trademark)} H-15 (triglycerides of saturated vegetable fatty acid; Riches-Nelson, Inc., New York), and has the following composition:

TABLE 5

| Ingredient | Amount |
|---|---|
| Compound of the present technology | 500 mg |
| Witepsol ® H-15 | balance |

The following synthetic and biological examples are offered to illustrate the present technology and are not to be construed in any way as limiting the scope of this the present technology. Unless otherwise stated, all temperatures are in degrees Celsius.

### Methods

In one aspect, the methods herein disclose where the method of treatment of a cancer decreases the expression level and/or activity of a Cdc7 in a subject.

In one aspect, the methods herein disclose where the cancer being treated is a cancer of epithelial origin.

In one aspect, the methods herein disclose where the cancer being treated is a cancer of gastrointestinal origin.

In one aspect, the methods herein disclose where the cancer being treated is a cancer of pulmonary origin.

In one aspect, the methods herein provide for an intravenous administration of one or more compounds of the present disclosure.

In one aspect, the methods herein provide for a subcutaneous administration of one or more compounds of the present disclosure.

In one aspect, the methods herein provide for selective Cdc7 inhibition to inhibition of the at least one other enzyme selected from the group consisting of: CLK1, CLK2, GSK3α, GSK3β, DYRK1B, Erk1, Erk2, PIM1, and p70S6K is at least 2-fold.

In one aspect, the methods herein provide for selective Cdc7 inhibition to inhibition of the at least one other enzyme selected from the group consisting of: CLK1, CLK2, GSK3α, GSK3β, DYRK1B, Erk1, Erk2, PIM1, and p70S6K is at least 4-fold.

In one aspect, the methods herein provide for selective Cdc7 inhibition to inhibition of the at least one other enzyme selected from the group consisting of: CLK1, CLK2, GSK3α, GSK3β, DYRK1B, Erk1, Erk2, PIM1, and p70S6K is at least 6-fold.

In one aspect, the methods herein provide for selective Cdc7 inhibition to inhibition of the at least one other enzyme selected from the group consisting of: CLK1, CLK2, GSK3α, GSK3β, DYRK1B, Erk1, Erk2, PIM1, and p70S6K is at least 8-fold.

In one aspect, the methods herein provide for treating a cancer that is associated with Cdc7 overexpression as compared to Cdc7 expression in a non-cancerous control cell, the method comprising administering an effective amount of a composition comprising one or more compounds herein disclosed to a subject in need thereof.

In one aspect, the methods herein provide for treating a cancer affected by Cdc7 activity, which method comprises administering to a subject an effective amount of one or more compounds of formula I-IV or a pharmaceutical composition comprising one or more compounds of formula I-IV and one or more pharmaceutically acceptable excipients.

In one aspect, the methods herein provide for inhibiting Cdc7, wherein the method comprises contacting cells with an effective amount of one or more compounds of formula I-IV or a pharmaceutical composition comprising one or more compounds of formula I-IV and one or more pharmaceutically acceptable excipients. In another aspect, the methods herein provide for selectively inhibiting Cdc7, wherein the method comprises contacting cells with an effective amount of one or more compounds of formula I-IV or a pharmaceutical composition comprising one or more compounds of formula I-IV and one or more pharmaceutically acceptable excipients while in the presence of at least one more enzymes selected from the group consisting of: CLK1, CLK2, GSK3α, GSK3β, DYRK1B, Erk1, Erk2, PIM1, and p70S6K.

In one aspect, the methods herein provide for inhibiting Cdc7, wherein the method comprises administering to a subject in need thereof an effective amount of one or more compounds of formula I-IV or a pharmaceutical composition comprising one or more compounds of formula I-IV and one or more pharmaceutically acceptable excipients.

In another aspect, a method is provided for prophylactic therapy or treatment of a subject having a cancer that is associated with, affected by, or that over-expresses a serine-threonine kinase and/or Cdc7 wherein said method comprises administering an effective amount of one or more compounds of formula I-IV disclosed herein to a subject in need thereof.

In some aspects, the present disclosure provides a method for treating a cancer selected from the group consisting of: colon cancer, blood cancer, and cervical cancer.

In some aspects, the present disclosure provides a method for treating a cancer selected from the group consisting of: colon cancer, blood cancer, cervical cancer, non-small cell lung cancer, pancreatic cancer, biliary tract cancer, bladder cancer, breast cancer, ovarian cancer, liver cancer and p53-mutated triple negative (ER−/PR−/Her2−) breast cancer.

In some aspects, the present disclosure provides a method for treating p53-mutated triple negative (ER−/PR−/Her2−) breast cancer with a compound or pharmaceutical composition disclosed herein.

In one aspect, the methods herein provide for treating lung cancer.

In one aspect, the methods herein provide for treating breast cancer. In one aspect, the methods herein provide for treating colon cancer.

In one aspect, the methods herein provide for treating ovarian cancer.

In one aspect, the methods herein provide for treating pancreatic cancer.

In another aspect, the present technology is directed to a method wherein one or more compounds of formula I-IV may be administered with other Cdc7 or serine-threonine inhibitor agents, such as anti-Cdc7 antibodies or antibody fragments, Cdc7 antisense iRNA, or other small molecule Cdc7 inhibitors, or in combination with other agents as described in detail herein.

In one aspect, the disclosure herein provides for a method of selectively inhibiting Cdc7, the method comprising contacting one or more compounds herein disclosed with Cdc7 and at least one more enzyme selected from the group consisting of: CLK1, CLK2, GSK3α, GSK3β, DYRK1B, Erk1, Erk2, PIM1, and p70S6K.

In one aspect, the disclosure herein provides for a method of selectively inhibiting Cdc7, wherein the inhibition is in the presence of 1 mM adenosine triphosphate (ATP).

In one aspect, the disclosure herein provides for a method of selectively inhibiting Cdc7, wherein the selectivity for Cdc7 inhibition to inhibition of the at least one other enzyme is at least 8-fold.

In one aspect, the disclosure herein provides for a method of selectively inhibiting Cdc7, wherein the compound inhibits Cdc7 with at least an IC₅₀ value of equal to or less than 1 μM.

In one aspect, the disclosure herein provides for a method according to any of the methods disclosed herein, of selectively inhibiting Cdc7, wherein the contacting takes place in a cell.

In one aspect, the disclosure herein provides for a method of inhibiting Cdc7 comprising contacting a cell with one or more compounds herein disclosed.

In one aspect, the disclosure herein provides for a method of inhibiting Cdc7, wherein the cell is a Colo-205 cancer cell.

In one aspect, the disclosure herein provides for a method of inhibiting Cdc7, wherein the cell is a LS174T cancer cell.

In one aspect, the disclosure herein provides for a method of inhibiting Cdc7, wherein the cell is a DoHH2 cancer cell.

In one aspect, the disclosure herein provides for a method of inhibiting Cdc7, wherein the cell is a HeLa cancer cell.

In one aspect, the disclosure herein provides for a method for treating a cancer that is associated with Cdc7 overexpression as compared to Cdc7 expression in a non-cancerous control cell, the method comprising administering an effective amount of a composition comprising one or more compounds herein disclosed to a subject in need thereof.

In one aspect, the disclosure herein provides for a method for treating a cancer that is associated with Cdc7 overexpression as compared to Cdc7 expression in a non-cancerous control cell, wherein the cancer is selected from the group consisting of colon cancer, blood cancer, and cervical cancer.

In one aspect, the disclosure herein provides for a method for treating a cancer that is associated with Cdc7 overexpression as compared to Cdc7 expression in a non-cancerous control cell, wherein the cancer is colon cancer.

In one aspect, the disclosure herein provides for a method for treating a cancer that is associated with Cdc7 overexpression as compared to Cdc7 expression in a non-cancerous control cell, wherein the subject is a mammal.

In one aspect, the disclosure herein provides for a method for treating a cancer that is associated with Cdc7 overexpression as compared to Cdc7 expression in a non-cancerous control cell, wherein the subject is a human.

In one aspect, the disclosure herein provides for the use of one or more compounds disclosed herein above for the manufacture of a medicament for treating cancer that is associated with Cdc7 overexpression as compared to Cdc7 expression in a non-cancerous control cell.

Cancers that associated with, affected by, or that over-expresses a serine-threonine kinase and/or Cdc7 include those selected from the group consisting of: colon cancer, blood cancer, cervical cancer, non-small cell lung cancer, pancreatic cancer, biliary tract cancer, bladder cancer, breast cancer, ovarian cancer, liver cancer and p53-mutated triple negative (ER−/PR−/Her2−) breast cancer.

The compounds of the present technology are useful in the diagnosis and treatment of a variety of cancers selected from the group consisting of: colon cancer, blood cancer, cervical cancer, non-small cell lung cancer, pancreatic cancer, biliary tract cancer, bladder cancer, breast cancer, ovarian cancer, liver cancer and p53-mutated triple negative (ER−/PR−/Her2−) breast cancer.

The amount of active compound administered will vary depending upon the disease treated, the mammalian species, and the particular mode of administration, etc. Suitable doses for the compounds of the present technology can be, for example, between 0.1 mg to about 1000 mg, between 1 mg to about 500 mg, between 1 mg to about 300 mg, or between 1 mg to about 100 mg per day. Such doses can be administered once a day or more than once a day, for example 2, 3, 4, 5 or 6 times a day, but preferably 1 or 2 times per day. In some embodiments, the total dosage for a 70 kg adult is in the range of 0.001 to about 15 mg per kg weight of subject per administration or 0.01 to about 1.5 mg per kg weight of subject per administration, and such therapy can extend for a number of days, a number of weeks or months, and in some cases, years. It will be understood, however, that the specific dose level for any particular subject will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

The following synthetic and biological examples are offered to illustrate the present technology and are not to be construed in any way as limiting the scope of this the present technology. Unless otherwise stated, all temperatures are in degrees Celsius.

### EXAMPLES

The compounds of the present technology can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will also employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

General Chemical and Biochemical Methods

Reagents and solvents were purchased from commercial sources and used without further purification. All reactions involving air- or moisture-sensitive reagents were performed under a nitrogen atmosphere unless otherwise noted. Microwave reactions were run in a Biotage Initiator set to normal power at the indicated temperature and were performed in sealed microwave reaction vessels. Silica gel chromatography refers to the use of an automated medium pressure liquid chromatography system (Teledyne ISCO or Yamazen Corp.) using prepacked silica gel cartridges with UV detection at 254 nm. Preparative reverse-phase HPLC (prep-HPLC) was performed on a Waters Autopurification system (dual triggered by target mass and UV 254 nm) using Imtakt Unison US-C18, 5 μm, 50 mm×20 mm I.D. column, eluting with a binary solvent system A and B using a gradient elution (A, 10 mM formic acid aq.; B, 10 mM formic acid in MeOH). All yields reported are isolated yield after removal of residual solvents. The purity of a purified compound was determined using Shimazu Prominence HPLC system by UV detection (215 nm) with collecting MS spectra (100-800 m/z scan) of the target peak. The separation method is shown as following. Column: Imtakt Cadenza, 3 μm, 50 mm×2.0 mm I.D. Mobile phase: acetonitrile in water (10 mM formic acid) from 10% to 90% with total 5 min run. Flow rate: 0.5 mL/min. ¹H-NMR spectra were recorded on Burker Ultra Shield™ 400 Plus; chemical shifts (δ) are reported relative to a signal of TMS. Data format of NMR spectra is as follows: chemical shift (δ ppm), multiplicity (s=singlet, br. s.=broad singlet, d=doublet, t=triplet, q=quartet, dd=doublet of doublets, dt=doublet of triplets, m=multiplet or overlapping), coupling constant (Hz), and integration. The synthesized compounds for biological assay have over 95% purity, otherwise noted in each synthesis experimental below.

### Modeling Study

Molecular modeling activities were performed using the suite of programs within the Discovery Studio release 2.1 (Accelrys Software). Homology models of Cdc7 were built from a Cdc7Δ212-368/CK2α alignment and structural data of CK2α (pdb: 3bw5). The compound 3 was then flexibly docked into the active site of the homology models using Flexible Docking Module with a standard parameter setup

Cdc7/ASK Enzymatic Assay

Measurement of Cdc7 kinase activity was carried out using MSA assay kit (QuickScout™ Screening Assist Kit, Carna Biosciences, Inc.). Assay buffer (20 mM HEPES, 0.01% Triton X-100™, 2 mM dithiothreitol, pH 7.5) was used to prepare a substrate mixture solution comprising 4 μM of kinase-reaction substrate (FITC-labeled MCM2 peptide), 40 mM MgCl₂, and 4 times concentrations of final targeted ATP (5, 100, and 1,000 μM). The enzyme supplied in the kit (human Cdc7/human ASK complex protein) was diluted into the assay buffer to prepare a 7 nM solution (enzyme solution). The compound stock solution was prepared by diluting a 10 mM DMSO solution into the assay buffer (final 4% DMSO). The kinase reaction was performed by mixing of 5 μL of the compound solution, 5 μL of the substrate mixture solution, and 10 μL of the enzyme solution in a 384-well plate. For the pre-incubation assay, the compound solution and the enzyme solution were pre-mixed and incubated at ambient temperature for 30 min before adding into the substrate mixture solution to start enzymatic reaction. After the reaction at 25° C. for 5 hours, 60 μL of the termination buffer (supplied in the kit) was added to stop the reaction. The phosphorylation of substrate was measured using a LabChip EZ Reader II system (Caliper Life Sciences). The percent inhibition (%) by a tested compound was calculated according to the following formula:

Percent inhibition (%)=(1−(C−A)/(B−A))×100

A represents P/(P+S) for a blank well, B represents P/(P+S) for a solvent well, and C represents P/(P+S) for a compound-added well. S: the peak heights of the separated substrate, P: phosphorylated substrate.

The IC₅₀ value of tested compound was calculated by regression analysis of the percent inhibition values versus the (logarithmic) concentration of the tested compound.

### In Vitro Metabolic Stability Study

NADPH regeneration system (13 mM NADP, 33 mM Glucose-6-phospate, 33 mM MgCl₂, and 4 U/mL of Glucose-6-phosphate dehydrogenase) and liver microsomes (1 mg/mL of HLM (BD Gentest, lot 18888) or MLM (BD Gentest, lot 31197)) in 0.1 M phosphate buffer (pH 7.4) were pre-incubated separately at 37° C. for 20 min before mixing with 1:4 ratio. 50 μL of the mixed solution was added to 50 μL of the test compound (2 μM) to initiate the reaction at 37° C. (final conc. 1 μM test compound, 0.4 mg/mL liver microsomes, and 1.3 mM NADPH). After 30 min, the reactions were stopped by adding 300 μL of cold acetonitrile. For 0 min samples, ice-cold acetonitrile (with system suitability standard) was treated before mixing the test compound and microsomes. The sample were vortexed well and then centrifuged at 3500 rpm at 15° C. for 20 min. 110 μL of supernatant was mixed with 110 μL of water and quantitated by LC-MS/MS workstation (Shimazu Corp.).

Western Blotting

Human colon adenocarcinoma cell line Colo-205 was obtained from American Type Culture Collection (ATCC, Manassas Va.). Cells were grown in RPMI1640 supplemented with 10% FBS and 1% penicillin/streptomycin at 37° C., 5% CO₂ and 95% humidity. Antibodies against Phospho-Histone H2A.X (Ser139) (no. 2577), Cleaved PARP (Asp214) (no. 9541) and MCM2 (no. 3619) were obtained from Cell signaling Technology. Anti-phospho-MCM2 (Ser53) (no. ab109133) and anti-β-actin (no. ab6276) antibodies were obtained from Abcam.

Cells were seeded in T75 flasks (cell density, 2.25×10⁶ cells/13.5 mL) and then incubated at 37° C. overnight. The next day, 1.5 mL of test compound solution (10× conc. of final conc.) was added to the flask, and incubated at 37° C. for 48 h. After washing with cold D-PBS, cells were scraped and lysed using a lysis buffer [RIPA Buffer (×1) containing 1% Phosphatase inhibitor Cocktail 3, 1% Phosphatase inhibitor Cocktail, and 1 mM phenylmethylsulfonyl fluoride], and the obtained samples were analyzed by western blotting. Band detection was performed using Chemi-Lumi One Super (Nacalai tesque Inc.) according to the manufacturer's protocol. Each band was detected by chemiluminescence using a CCD camera (ImageQuant LAS 500, GE healthcare Ltd.).

Cell Cycle Analysis

Colo-205 cells (5×10⁵) were seeded in six-well plates and treated with different concentrations of a test compound. After 48 h, the cells were harvested and suspended in 0.5 mL of D-PBS. The cells were then fixed by adding 1 mL of ice-cold ethanol, and the samples were kept at −30° C. The cells were resuspended in 200 μL PI solution (20 μg/mL propydium iodide and 100 μg/mL RNaseA of D-PBS), and incubated in the dark for 1 h at room temperature. Cell cycle analyses were performed using FACS Calibur flow cytometer (BD Biosciences).

### Example 1

Compound SAR Study & Inhibitory Data.

To study the structure-activity relationship (SAR) of the 7-azaindolyl furanone derivatives more clearly, enzymatic assays of Cdc7 were performed at 5 μM ATP concentration. The initial compound 3 showed 21-fold stronger inhibitory potency (IC₅₀=3.3 nM) than inhibition at 10 μM ATP concentration, suggesting that this compound, and others closely related having the 7-azaindolyl furanone core structure, are ATP-competitive inhibitor.

Initially, we synthesized compounds having different X and R¹ groups to examine the impact of the furanone ring and azaindole ring on the potency. As shown in Table 1, replacing O with S resulted in retention of some potency (compound 4). Moreover, incorporation of phenol (6) or methylenedioxyphenyl (7) groups as hinge binders also retained some potency (IC₅₀=1200 and 3200 nM, respectively). Finally, introduction of Cl at the 5-position in the azaindole ring (5) did not deteriorate its inhibitory potency significantly, as was predicted from the binding model (IC₅₀=44 nM). These results suggest that the furanone ring and the azaindole ring are important and can be further modified or optimized with different functional groups for inhibitory activity.

### [Table 6]

TABLE 1
Effects of furanone and azaindole modification on Cdc7 inhibitory
activities

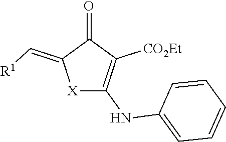

| | | | Cdc7 IC₅₀ |
|---|---|---|---|
| Cpd | R¹ | X | (nM)^{a} |
| 3 | 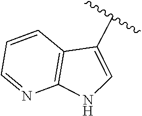 [SMILES: *c1c[nH]c2ncccc12] | O | 3.8 |
| 4 | 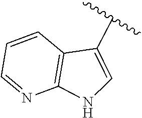 [SMILES: *c1c[nH]c2ncccc12] | S | >10,000 |
| 5 | 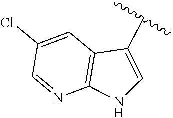 [SMILES: *c1c[nH]c2ncc(Cl)cc12] | O | 44 |
| 6 | 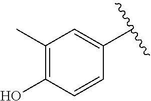 [SMILES: *c1ccc(O)c(C)c1] | O | 1,200 |
| 7 | 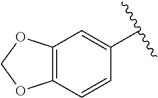 [SMILES: *c1ccc2c(c1)OCO2] | O | 3,200 |

^{a}IC₅₀ values are reported as the mean of duplicated assays in the presence of 5 μM of ATP.

Because the ester was expected to form important hydrogen bond with Lys90, an SAR study for the ester group was performed (Table 2). As expected, compounds having hydrogen bond accepting oxygen such as OMe (8) and OH (9) maintained potency (IC₅₀=3.3 and 9.9 nM, respectively). However, a significant decrease in potency was observed when hydrogen bond donor functional groups were installed (10 and 11). However, the introduction of a piperidine functional group (12) resulted in a modest decrease in potency (IC₅₀=890 nM), suggesting that the piperidine ring interacts with Cdc7. Generally esters are labile and can be easily hydrolyzed to the corresponding carboxylic acid. To our surprise, the ethyl ester of 3 was very stable even under strong basic conditions, and it required an extremely strong basic condition (50% w/v KOH aq., reflux) to hydrolyze this ester.

### [Table 7]

TABLE 2
Effects of R² on Cdc7 inhibitory activities

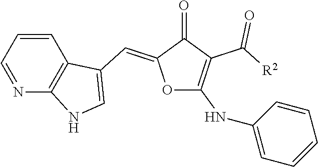

| Cpd | R² | Cdc7 IC₅₀ (nM)^{a} |
|---|---|---|
| 3 | OEt | 3.8 |
| 8 | OMe | 3.3 |
| 9 | OH | 9.9 |
| 10 | NH₂ | >10,000 |
| 11 | NHEt | 1,600 |
| 12 | 1-Piperidinyl | 890 |

^{a}IC₅₀ values are reported as the mean of duplicated assays in the presence of 5 μM of ATP.

The effects of substituents R⁴ at the benzene ring are shown in Table 3. Surprisingly, both electron withdrawing and electron donating aromatic ring substitutions were well tolerated, and remarkably, retained high inhibitory potency.

Compound 3 displayed moderate metabolic stability in human liver microsome, but it was rapidly metabolized in mice (Table 3). It was hypothesized, based on modeling data, that the unsubstituted benzene ring of compound 3 could be metabolized rapidly. Thus, the metabolic stabilities of substituted benzene derivatives were evaluated.

### [Table 8]

TABLE 3
Effects of substitution at benzene ring on Cdc7 inhibitory activities
and liver microsomes stabilities

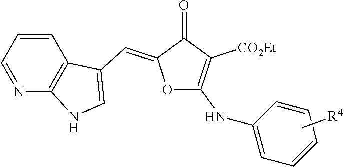

| | | Cdc7 | HLM^{b} | MLM^{b} |
|---|---|---|---|---|
| Cpd | R⁴ | IC₅₀ (nM)^{a} | % | % |
| 3 | H | 3.8 | 23 | 7.9 |
| 13 | 2-C1 | 2.4 | 67 | 58 |
| 14 | 3-C1 | 12 | — | — |
| 15 | 4-C1 | 6.7 | — | — |
| 16 | 2-MeO | 12 | — | — |
| 17 | 3-MeO | 29 | — | — |
| 18 | 4-MeO | 4.9 | 33 | 5.1 |
| 19 | 2-F | 2.4 | 30 | 10 |
| 20 | 4-F | 4.3 | 52 | 25 |
| 21 | 2-Me | 3.8 | 53 | 24 |
| 22 | 4-Me | 5.9 | — | — |
| 23 | 2,4-di-F | 2.5 | 57 | 12 |
| 24 | 2,4-di-Me | 3.9 | 63 | 30 |

^{a}IC₅₀ values are reported as the mean of duplicated assays in the presence of 5 μM of ATP.
^{b}Remaining % of parent compounds after 30 min treatment of liver microsomes. HLM and MLM: human and mouse liver microsomes.

Replacement of the benzene ring with other heterocycles could provide an insight into structural requirement of the binding site (Table 4). Gratifyingly, stereoelectronic divergent 3- And 4-pyridines (26 and 27) showed high potency comparable to that of the benzene analog 3 (IC₅₀=8.0 and 3.5 nM, respectively). Insertion of alkyl chains between phenyl ring and NH resulted in some decrease in potency (32 and 33).

### [Table 9]

TABLE 4
Effect of R³ position on Cdc7 inhibitory activities

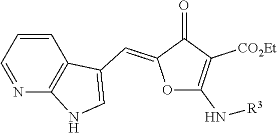

| | | Cdc7 |
|---|---|---|
| Cpd | R³ | IC₅₀ (nM)^{a} |
| 3 | Ph | 3.8 |
| 25 | 2-Py | 30 |
| 26 | 3-Py | 8.0 |
| 27 | 4-Py | 3.5 |
| 28 | 5-Pyrimidinyl | 18 |
| 29 | 3-Pyrazolyl | 10 |
| 30 | 6-Quinolinyl | 36 |
| 31 | 6-Indazoyl | 4.0 |
| 32 | Benzyl | 23 |
| 33 | Phenethyl | 30 |

^{a}IC₅₀ values are reported as the mean of duplicated assays in the presence of 5 μM of ATP.

### Example 2

ATP Dependency and Pre-Incubation Effects of 13 on Cdc7 Inhibition

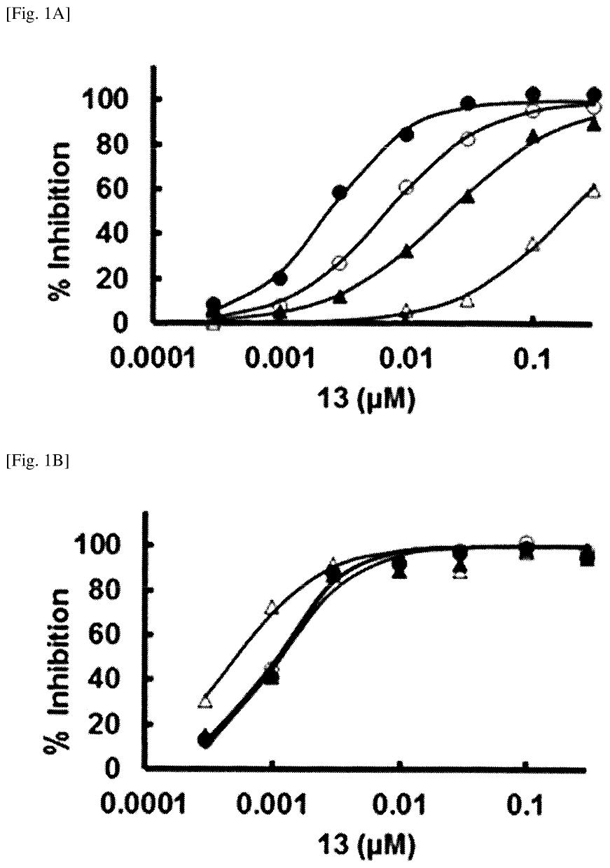

As compound 13 showed improved metabolic stabilities in mice and human, this compound was selected for further evaluations. ATP competition assay was performed to determine whether compound 13 is ATP competitive or not. Dose response curves of compound 13 in the presence of various ATP concentrations are shown in FIG. 1a. Compound 13 displayed ATP-dependent proportional decreases in inhibitory potencies for Cdc7, suggesting that it is an ATP competitive inhibitor. The increase in ATP concentrations resulted in a dramatic increase in IC₅₀ values of 13 (IC₅₀=191 nM in the presence of 1 mM ATP). It has been reported that some kinase inhibitors show time-dependent inhibition due to slow on- and/or off-rate for binding. Therefore, we have examined a time dependency of Cdc7 inhibition by 13 with pre-incubation method. Namely, compound 13 was pre-incubated for 30 minutes with Cdc7 prior to the addition of the substrates mixture to start enzymatic reactions. Surprisingly, the dose response curves in the presence of various ATP concentrations were not changed, and the inhibitory potencies were sustained even in the presence of 1 mM ATP when 13 was pre-incubated with the enzyme (FIG. 1b). On the other hand, other chemotype of compound 1b did not show such pre-incubation effects (data not shown), suggesting that this pre-incubation effect was unique to the 7-azaindolyl furanone compounds we identified.

### Example 3

Comparison of Dissociation Rates from Cdc7

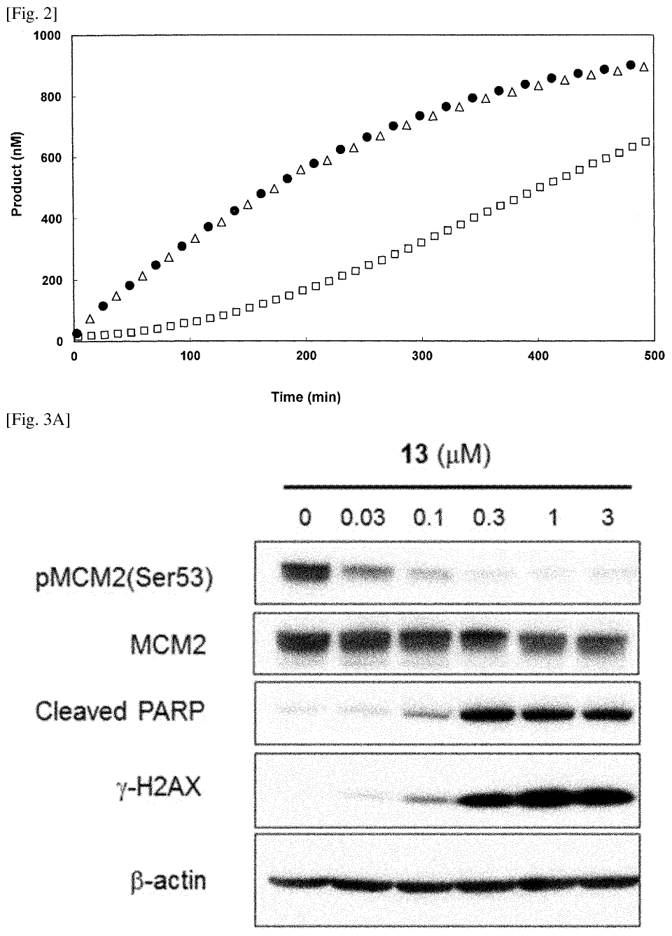

To better understand the observed time dependent inhibition of these compounds, compound 13 was subjected to the rapid dilution assay to study dissociation rate, and compared with compound 1. The inhibitor-enzyme mixture was rapidly diluted into the assay buffer containing the substrate and a high concentration of ATP, and then the phosphorylated product formation was monitored over 500 min of time course. As shown in FIG. 2, the curve of the product formation in the presence of compound 1 was almost the same as that of vehicle control. On the other hand, Cdc7 produced the phosphorylated product with much reduced rate after treatment with compound 13. We estimated the off-rate of compound 13 from Cdc7 (k_{off}) to be 3.5×10⁻³ min⁻¹ (t_{1/2}=197 min). These results suggested that compound 13 has a unique inhibitory mechanism that binds to Cdc7 in a reversible fashion but has a very slow off-rate.

### Example 4

Kinase Selectivity

To investigate the representative kinase selectivity of these compounds at physiological ATP levels, compound 13 was screened over a panel of 121 kinases in the presence of 1 mM ATP with preincubation method. Compound 13 showed an excellent selectivity profile with only 7% of the panel inhibited (>50% inhibition at 0.1 μM). IC₅₀ values of hit kinases were determined to confirm the selectivity of compound 13. As shown in Table 5, cross-reactivities with CLKs were observed albeit with selectivity in favor of Cdc7 (8-fold). In addition, the assays demonstrated that compound 13 has fair to good selectivity for other off-target hit kinases, with 61 to 655-fold selectivity.

### [Table 10]

TABLE 5
Kinase selectivity of compound 13

| Kinases | IC50 (nM)^{a} | Fold selectivity^{b} |
|---|---|---|
| Cdc7 | 0.6 | 1.0 |
| CLK1 | 5.0 | 8.3 |
| CLK2 | 5.3 | 8.8 |
| GSK3α | 37 | 61 |
| GSK3β | 51 | 85 |
| DYRK1B | 53 | 88 |
| Erk2 | 61 | 102 |
| PIM1 | 77 | 129 |
| Erk1 | 278 | 463 |
| p70S6K | 399 | 665 |

^{a}IC₅₀ values are reported as the mean of duplicated assays.
^{b}Data are expressed as fold selectivity of the IC₅₀ value for each kinase versus Cdc7.

### Example 5

Cellular Effects

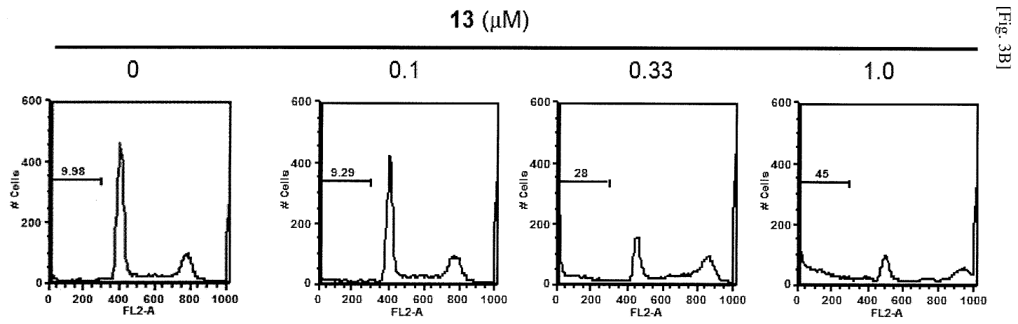

The results of the studies for the enzyme inhibition and the kinase selectivity profiling confirmed that the model used to identify these compounds was accurate and that 13 was predicted to be a potent and selective inhibitor of Cdc7 in cells. This compound was subjected to cellular experiments. To investigate the effects of selective Cdc7 inhibition in cancer cells, we evaluated the ability of compound 13 to inhibit phosphorylation of MCM2 and the functional consequences in Colo-205 cells. As shown in FIG. 3a, compound 13 potently inhibited phosphorylation at Ser53 of MCM2 even at 0.1 μM. It has been reported that knockdown of Cdc7 was shown to cause cell death in cancer cells, because it results in cell cycle progression through a defective S phase and the subsequent DNA strand breakage that leading to p53-independent apoptotic cell death. In 13-treated cells, the DNA strand breakages were observed in response to the MCM2 inhibition, by analyzing phospho-H2AX (γ-H2AX), a well-established marker of DNA double strand breaks. Dose-dependent increases in cleaved PARP, the apoptosis marker were also observed, suggesting the observed cell death was elicited by selective inhibition of Cdc7 by 13. Compound 13-treated cells were then subjected to cell cycle analysis (FIG. 3b). DNA contents in Colo-205 cells were analyzed by flow cytometry after 48 hours of compound treatment. Marked decreases in G1 phase peak and significant increase in sub-G1 population were observed after treatment with 0.33 and 1 μM compound 13, as indicative of cell death. This is consistent with the previous report that Cdc7 knockdown in cancer cells induced DNA double strand breaks and cell death. Anti-proliferation activity of compound 13 was determined also in Colo-205 cells. Treatment of Colo-205 cells with compound 13 led to reduction of the cell proliferation with an IC₅₀ value of 0.17 μM, which was consistent with the results of apoptosis marker studies. On the other hand, weaker effect in HEL299, derived from a human embryo lung tissue, was observed (IC₅₀=1.2 μM), further supporting the selective inhibition of Cdc7 by 13 in vivo.

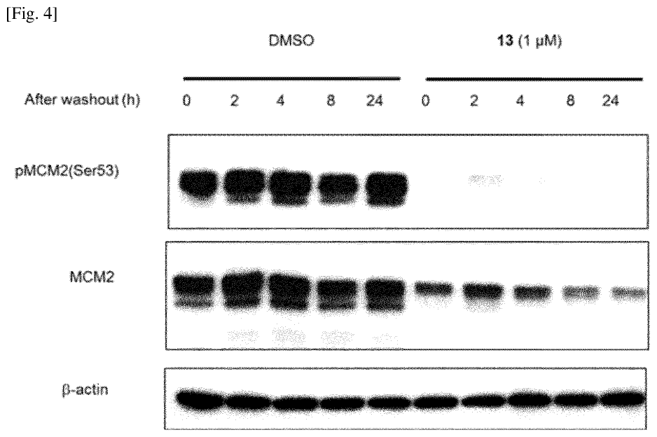

Next, we investigated the recovery of substrate phosphorylation in cells after inhibitor washout to see whether the slow-off rate of inhibitor affects the recovery of phosphorylation. Colo-205 cells were treated for 48 hours with 13, and then, the inhibitor was washed out with compound-free media. The cells were lysed at the indicated time, and the phosphorylation status of MCM2 was analyzed by western blotting. As shown in FIG. 4, the phosphorylation of MCM2 in 13-treated cells was inhibited even 24 hours after washout. These results indicate that the slow off-rate observed in 13 may reflect the duration of inhibitor efficacy in vivo.

The half-life of compound 13 after intravenous administration in mice was only 0.38 h, which hampered further investigations of this compound in vivo. Accordingly, the study suggests that substituted compounds, especially those substitutions on the aryl moieties will further improve the efficacy of the inhibitor with improved first-metabolite stability.

### Example 6

Synthesis of the Compounds

Compounds of the present disclosure were prepared by a Knoevenagel condensation reaction between the 7-azaindole-3-aldehyde and commercially available Knoevenagel donors using piperidine as catalyst (Scheme 1A). For the exploration of hinge binders R¹, the corresponding aldehydes were employed as Knoevenagel acceptors for the condensation reaction with furanone 35 to yield the desired compounds (5-7) as shown in Scheme 1B. For the exploration of hinge binders R¹, the corresponding aldehydes were employed as Knoevenagel acceptors for the condensation reaction with furanone 35 to yield the desired compounds (5-7) as shown in Scheme 1B.

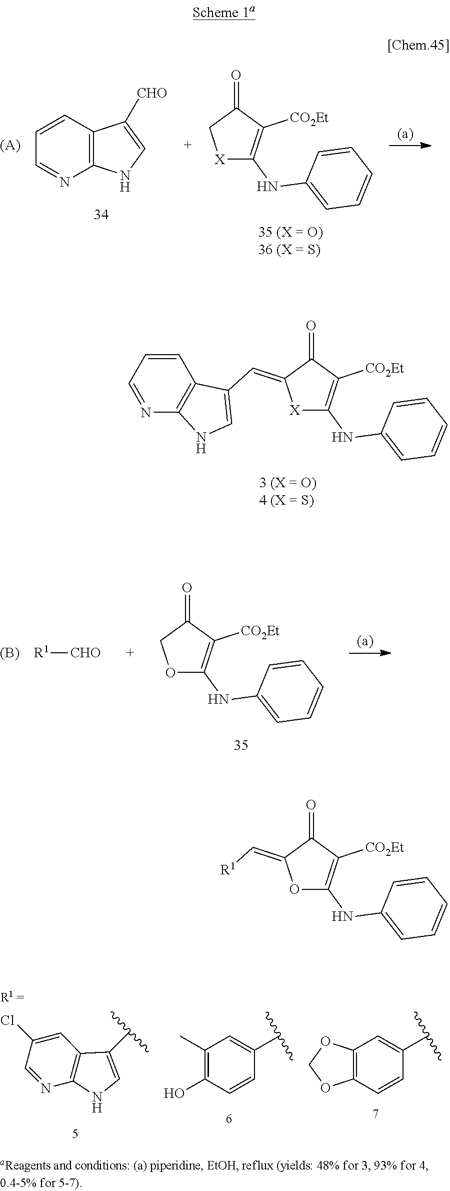

The general procedure for the replacement of R² by various groups is shown in Scheme 2. Methyl ester 8 was obtained analogously to ethyl ester 3 from the furanone intermediate 39 which was prepared from methyl 4-chloroacetoacetate 37 and phenyl isocyanate 38 (Scheme 2A). Compound 9 (Scheme 2B) was obtained by hydrolysis of compound 3 under extremely strong basic condition (50% w/v KOH aq., reflux). Surprisingly the ethyl ester in 3 was very stable even under conventional ester hydrolysis conditions such as acidic (2N HCl aq./EtOH, 60° C.) or basic (2N NaOH aq./EtOH, 60° C.) conditions. The amide analogs (10-12) were prepared directly by aminolysis of ethyl ester 3 with appropriate amines (Scheme 2C).

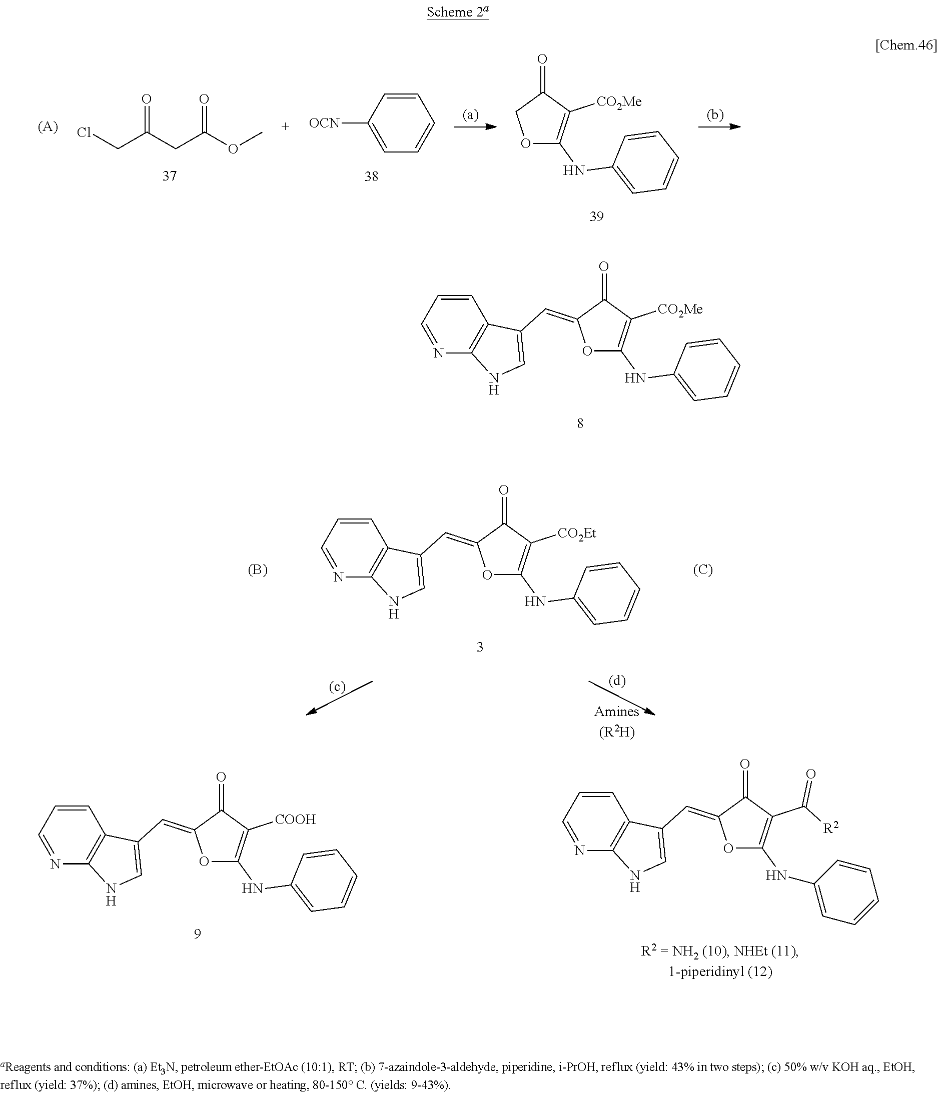

The modification of the R³ moiety was performed by two methods (method A and B). Commercially available isocyanate was treated with ethyl 4-chloroacetoacetate 40 to furnish furanone intermediate 43 in high yield (method A). Alternatively, furanone 43 was obtained by one-pot methodology from diethyl malonate 41 (method B). Namely, diethyl malonate 41 was treated with sodium hydride, and allowed to react with chloroacetyl chloride 42, followed by treating with an appropriate amine to give the corresponding furanone intermediate 43 in one-pot reaction. Completion of the synthesis was achieved as described in Scheme 1A to afford the desired products (13-33). In some cases, acidic conditions were employed in Knoevenagel condensation to achieve a faster reaction, and the product was isolated as the hydrochloride salt (32 and 33).

The furanone analogs synthesized in this report were isolated as a single isomer by NMR spectra unless otherwise noted in the Experimental Section.

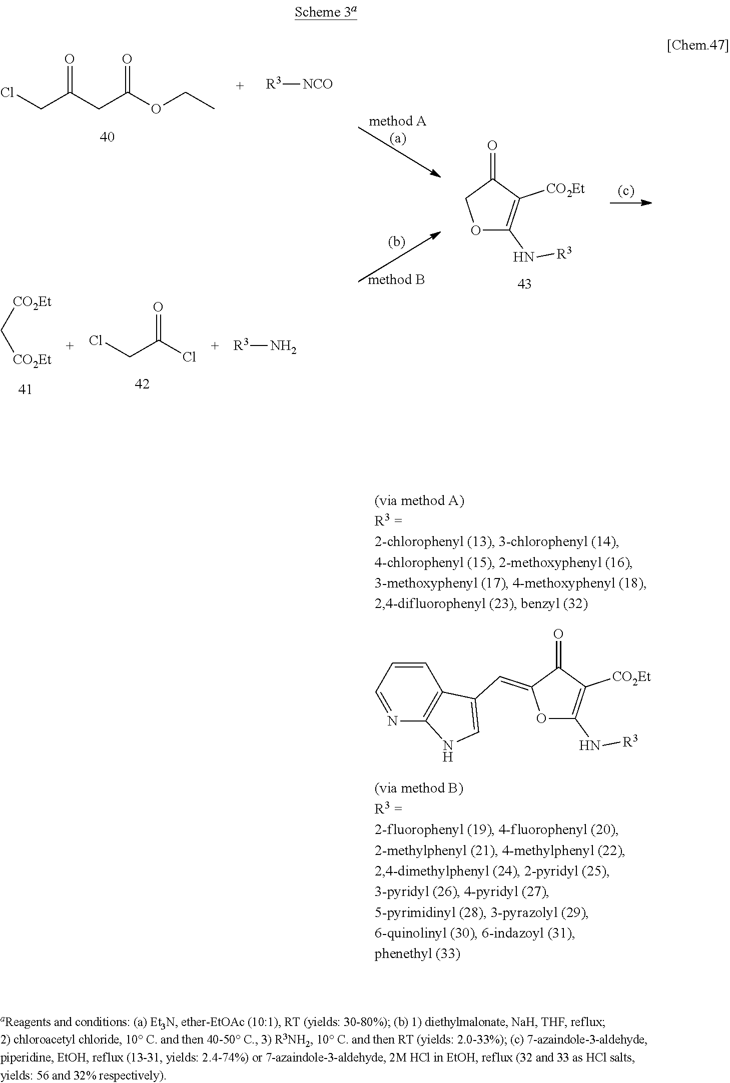

4.1.1 General Procedure of Knoegenavel Reactions (GP-I): Ethyl

### 5-((1H-pyrrolo[2,3-b]pyridin-3-yl)methylene)-4-oxo-2-(phenylamino)-4,5-dihydrofuran-3-carboxylate (3)

To a solution of 7-azaindole-3-carboxaldehyde (34, 0.10 g, 0.70 mmol) and ethyl 4-oxo-2-(phenylamino)-4,5-dihydrofuran-3-carboxylate (35, 0.18 g, 0.70 mmol) in ethanol (3.0 mL), piperidine (0.083 mL, 0.84 mmol) was added at ambient temperature. The mixture was heated at reflux for 12 h. After cooling to ambient temperature, the precipitate was collected by filtration, washed successively with cold ethanol and isopropyl ether, and then dried to afford 3 (0.13 g, 48%) as a colorless solid. ¹H NMR (400 MHz, DMSO-d⁶) δ 12.30 (br. s., 1H), 10.58 (s, 1H), 8.23 (dd, J=1.3, 4.6 Hz, 1H), 7.98 (d, J=7.3 Hz, 1H), 7.79 (d, J=2.8 Hz, 1H), 7.52-7.64 (m, 4H), 7.43-7.51 (m, 1H), 6.80-6.99 (m, 2H), 4.28 (q, J=7.0 Hz, 2H), 1.30 (t, J=7.0 Hz, 3H). ¹³C NMR (101 MHz, DMSO-d⁶) δ 176.18, 171.32, 163.98, 149.20, 144.20, 142.90, 135.62, 131.12, 129.66, 128.82, 127.74, 126.14, 118.37, 117.01, 106.95, 103.04, 88.31, 59.64, 40.41, 40.20, 39.99, 39.78, 39.58, 15.03. MS m/z 376 [M+H]⁺.

4.1.2 Ethyl

### 5-((1H-pyrrolo[2,3-b]pyridin-3-yl)methylene)-4-oxo-2-(phenylamino)-4,5-dihydrothiophene-3-carboxylate (4)

Compound 4 was synthesized from 7-azaindole-3-carboxaldehyde (34) and ethyl 4-oxo-2-(phenylamino)-4,5-dihydrothiophene-3-carboxylate (36) utilizing similar reaction conditions as described in GP-I as an yellow solid (257 mg, 93%). ¹H NMR (400 MHz, DMSO-d⁶) δ 12.44 (s, 1H), 11.27 (s, 1H), 8.34 (dd, J=4.7, 1.5 Hz, 1H), 8.28 (dd, J=8.1, 1.6 Hz, 1H), 7.89 (d, J=0.7 Hz, 1H), 7.72 (d, J=2.8 Hz, 1H), 7.60-7.50 (m, 4H), 7.48-7.39 (m, 1H), 7.22 (dd, J=7.9, 4.7 Hz, 1H), 4.29 (q, J=7.1 Hz, 2H), 1.31 (t, J=7.1 Hz, 3H). MS m/z 392 [M+H]⁺.

4.1.3 Ethyl

### 5-((5-chloro-1H-pyrrolo[2,3-b]pyridin-3-yl)methylene)-4-oxo-2-(phenylamino)-4,5-dihydrofuran-3-carboxylate (5)

Step 1. To a solution of 5-chloro-7-azaindole (0.50 g, 3.3 mmol) in acetic acid (5.0 mL), hexamethylenetetramine (0.69 g, 4.9 mmol) was added at ambient temperature. The mixture was heated at reflux for 8 h. After cooling to ambient temperature, the reaction mixture was diluted with water, extracted with ethyl acetate twice. The organic layer was washed successively with water and brine, dried over sodium sulfate and concentrated. The residue was purified by chromatography on silica gel (hexane/ethyl acetate) to afford 5-chloro-7-azaindole-3-carboxyaldehyde (0.13 g, 22%) as a colorless solid. ¹H NMR (400 MHz, DMSO-d⁶) δ 10.01 (s, 1H), 9.77 (br. s, 1H), 8.61 (d, J=2.0 Hz, 1H), 8.37 (d, J=2.1 Hz, 1H), 7.99 (d, J=2.6 Hz, 1H). MS m/z 181 [M+H]⁺.

Step 2. Compound 5 was synthesized from 5-chloro-7-azaindole-3-carboxaldehyde prepared in Step 1 and ethyl 4-oxo-2-(phenylamino)-4,5-dihydrofuran-3-carboxylate (35) as an yellow solid (2.0 mg, 4.6%), utilizing similar reaction conditions as described in GP-I with prep-HPLC purification. ¹H NMR (400 MHz, DMSO-d⁶) δ 12.52 (br. s., 1H), 10.58 (s, 1H), 8.42 (d, J=2.3 Hz, 1H), 8.26 (d, J=2.3 Hz, 1H), 7.71 (d, J=2.5 Hz, 1H), 7.55-7.63 (m, 2H), 7.51 (t, J=7.8 Hz, 2H), 7.35-7.44 (m, 1H), 7.00 (s, 1H), 4.28 (q, J=7.0 Hz, 2H), 1.30 (t, J=7.2 Hz, 3H). MS m/z 410 [M+H]⁺.

4.1.4 Ethyl

### 5-(4-hydroxy-3-methylbenzylidene)-4-oxo-2-(phenylamino)-4,5-dihydrofuran-3-carboxylate (6)

Compound 6 was synthesized from 4-hydroxy-3-methylbenzaldehyde and ethyl 4-oxo-2-(phenylamino)-4,5-dihydrofuran-3-carboxylate (35) as a brown solid (2.9 mg, 4.8%, purity 91%), utilizing similar reaction conditions as described in GP-I with prep-HPLC purification. ¹H NMR (400 MHz, DMSO-d⁶) δ 9.88 (s, 1H), 7.48-7.59 (m, 4H), 7.42 (d, J=2.2 Hz, 1H), 7.34-7.40 (m, 1H), 7.27 (dd, J=8.5, 2.3 Hz, 1H), 6.73 (d, J=8.3 Hz, 1H), 6.43 (s, 1H), 4.25 (q, J=7.1 Hz, 2H), 2.98-3.04 (m, 1H), 2.00 (s, 3H), 1.28 (t, J=7.1 Hz, 3H). MS m/z 366 [M+H]⁺.

4.1.5 Ethyl

### 5-(benzo[d][1,3]dioxol-5-ylmethylene)-4-oxo-2-(phenylamino)-4,5-dihydrofuran-3-carboxylate (7)

Compound 7 was synthesized from piperonal and ethyl 4-oxo-2-(phenylamino)-4,5-dihydrofuran-3-carboxylate (35) as a colorless solid (0.25 mg, 0.44%), utilizing similar reaction conditions as described in GP-I with prep-HPLC purification. ¹H NMR (400 MHz, DMSO-d⁶) δ 10.65 (s, 1H), 7.53-7.59 (m, 2H), 7.45-7.52 (m, 2H), 7.31-7.41 (m, 1H), 7.16-7.22 (m, 2H), 6.91 (d, J=8.0 Hz, 1H), 6.48-6.58 (m, 1H), 6.05 (s, 2H), 4.25 (q, J=7.1 Hz, 2H), 1.28 (t, J=7.0 Hz, 3H). MS m/z 380 [M+H]⁺.

4.1.6 Methyl

### 5-((1H-pyrrolo[2,3-b]pyridin-3-yl)methylene)-4-oxo-2-(phenylamino)-4,5-dihydrofuran-3-carboxylate (8)

Step 1. Methyl 4-oxo-2-(phenylamino)-4,5-dihydrofuran-3-carboxylate (39). To a mixed solution of methyl 4-chloroacetoacetate (3.7 mL, 30 mmol) and phenyl isocyanate (3.6 g, 33 mmol) in petroleum ether/ethyl acetate (50 mL/5.0 mL) that was cooled in an ice bath, triethylamine (4.8 g, 34 mmol) was added dropwise. The reaction mixture was stirred at ambient temperature for 1 h. The reaction suspension was diluted with water, 2N hydrochloric acid solution, and ether, and the solid was collected by filtration and washed successively with water and diethyl ether, and then dried to afford the titled compound (6.3 g, crude) as a colorless solid. MS m/z 234 [M+H]⁺.

Step 2. Compound 8 was synthesized from 7-azaindole-3-carboxaldehyde and methyl 4-oxo-2-(phenylamino)-4,5-dihydrofuran-3-carboxylate (39) prepared in Step 1 utilizing similar reaction conditions as described in GP-I as a colorless solid (31 mg, 43%). ¹H NMR (400 MHz, DMSO-d⁶) δ 12.29 (br. s., 1H), 10.59 (s, 1H), 8.23 (dd, J=1.4, 4.6 Hz, 1H), 7.98 (d, J=7.5 Hz, 1H), 7.77 (d, J=2.5 Hz, 1H), 7.52-7.64 (m, 4H), 7.44-7.51 (m, 1H), 6.93 (s, 1H), 6.89 (dd, J=4.6, 7.9 Hz, 1H), 3.77 (s, 3H). MS m/z 362 [M+H]⁺.

4.1.7

### 5-((1H-Pyrrolo[2,3-b]pyridin-3-yl)methylene)-4-oxo-2-(phenylamino)-4,5-dihydrofuran-3-carboxylic acid (9)

To a solution of compound 3 (50 mg, 0.13 mmol) in ethanol (1.0 mL), 50% potassium hydroxide solution (0.5 mL, 0.13 mmol) was added at ambient temperature. The mixture was heated at reflux for 1 h. After cooling to ambient temperature, the precipitate was collected by filtration, and washed with ethanol. The crude material was dissolved in water (0.5 mL) and tetrahydrofuran (0.5 mL), and then 2M hydrochloric acid (0.023 mL, 0.045 mmol) was added and the mixture was stirred for 30 min. The precipitate was collected by filtration, washed successively with water and diethyl ether, and dried to afford compound 9 (0.012 g, 26%) as a pale yellow solid. ¹H NMR (400 MHz, DMSO-d⁶) δ 12.11 (br. s., 2H), 8.21 (dd, J=1.3, 4.5 Hz, 1H), 8.06 (d, J=7.8 Hz, 1H), 7.73 (d, J=2.3 Hz, 1H), 7.38-7.49 (m, 2H), 7.32 (d, J=7.5 Hz, 2H), 7.19-7.27 (m, 1H), 6.91 (dd, J=4.6, 7.9 Hz, 1H), 6.66 (s, 1H). NH proton of the aniline is missing. MS m/z 348 [M+H]⁺.

4.1.8

### 5-((1H-Pyrrolo[2,3-b]pyridin-3-yl)methylene)-4-oxo-2-(phenylamino)-4,5-dihydrofuran-3-carboxamide (10)

The suspension of compound 3 (20 mg, 0.053 mmol) in 7 M ammonia in MeOH (1 ml, 7.00 mmol) and THF (0.7 ml) was heated under MW irradiation at 80° C. for 15 min. The resulting pale yellow solution was concentrated in vacuo. The residue was suspended in CHCl₃-MeOH and applied to silica gel chromatography (CHCl₃/MeOH=100/0 to 90/10, two peaks). The fractions of an earlier peak were collected and concentrated in vacuo to afford compound 10 (1.7 mg, 9.2%, purity 90%) as a colorless solid. ¹H NMR (400 MHz, DMSO-d⁶) δ 12.22 (s, 1H), 10.11 (s, 1H), 8.46 (dd, J=8.0, 1.6 Hz, 1H), 8.30 (dd, J=4.6, 1.6 Hz, 1H), 8.14 (s, 2H), 7.96 (s, 1H), 7.47-7.55 (m, 2H), 7.35-7.46 (m, 3H), 7.19 (dd, J=7.9, 4.7 Hz, 1H), 6.75 (d, J=0.6 Hz, 1H). MS m/z 347 [M+H]⁺.

4.1.9

### 5-((1H-Pyrrolo[2,3-b]pyridin-3-yl)methylene)-N-ethyl-4-oxo-2-(phenylamino)-4,5-dihydrofuran-3-carboxamide (11)

The suspension of compound 3 (20 mg, 0.053 mmol) in 70% w/v ethylamine in H₂O (50 μl, 0.62 mmol) and THF (1.0 ml) was heated at 80° C. for 7 h. The reaction mixture was concentrated in vacuo and purified by prep-HPLC to afford compound 11 (0.84 mg, 3.2% yield, regioisomer mixture=3:1) as a colorless solid. ¹H NMR (400 MHz, DMSO-d⁶) δ 12.20 (br. s., 0.75H), 11.64 (br. s., 0.25H), 10.07 (br. s., 0.75H), 9.78 (br. s., 0.25H), 8.39-8.55 (m, 1H), 8.29 (dd, J=4.5, 1.3 Hz, 0.75H), 8.17-8.25 (m, 0.25H), 7.93 (s, 0.75H), 7.67 (s, 0.25H), 7.20-7.50 (m, 6H), 7.18 (dd, J=7.9, 4.6 Hz, 0.25H), 7.08 (dd, J=7.8, 4.8 Hz, 0.25H), 6.70 (br. s., 1H), 2.75-3.10 (m, 2H), 1.07 (t, J=7.2 Hz, 3H). MS m/z 375 [M+H]⁺.

4.1.10

### 2-((1H-Pyrrolo[2,3-b]pyridin-3-yl)methylene)-5-(phenylamino)-4-(piperidine-1-carbon yl)furan-3(2H)-one (12)

The suspension of compound 3 (20 mg, 0.053 mmol) in piperidine (50 μl, 0.053 mmol) and THF (1.0 ml) was heated under MW irradiation at 150° C. for 15 min. The reaction mixture was concentrated in vacuo and purified by prep-HPLC to afford compound 12 (4.8 mg, 21%) as an yellow solid. ¹H NMR (400 MHz, DMSO-d⁶) δ 12.04 (br. s., 1H), 9.89 (s, 1H), 8.35 (d, J=7.3 Hz, 1H), 8.25 (dd, J=4.5, 1.5 Hz, 1H), 7.78 (d, J=2.3 Hz, 1H), 7.25-7.36 (m, 2H), 7.01-7.18 (m, 4H), 6.44 (s, 1H), 3.51 (br. s., 4H), 1.70 (br. s., 5H), 1.41-1.61 (m, 1H). MS m/z 415 [M+H]⁺.

4.1.11 General Procedure of Construction of Furanone Ester by Method A (GP-II)

Ethyl 2-[(2-chlorophenyl)amino]-4-oxo-4,5-dihydrofuran-3-carboxylate. To a mixed solution of ethyl 4-chloroacetoacetate (3.93 mL, 28.9 mmol) and 2-chlorophenyl isocyanate (3.83 g, 31.8 mmol) in petroleum ether/ethyl acetate (40 mL/4.0 mL) cooled with an ice bath, triethylamine (4.8 g, 34.4 mmol) was added dropwise. The reaction mixture was stirred at ambient temperature for 1 h. The reaction suspension was diluted with water, 2N hydrochloric acid solution and ether, and the solid was collected by filtration and washed successively with water and diethyl ether, and then dried to afford the titled compound (4.35 g, 53%) as a colorless solid. ¹H NMR (400 MHz, DMSO-d⁶) δ 10.50 (s, 1H), 7.73 (dd, J=1.51, 8.03 Hz, 1H), 7.63 (dd, J=1.25, 8.03 Hz, 1H), 7.44 (dt, J=1.51, 7.78 Hz, 1H), 7.34 (dt, J=1.51, 7.78 Hz, 1H), 4.72 (s, 2H), 4.24 (q, J=7.19 Hz, 2H), 1.26 (t, J=7.03 Hz, 3H). MS m/z 282 [M+H]⁺.

4.1.12 Ethyl

### 5-((1H-pyrrolo[2,3-b]pyridin-3-yl)methylene)-2-((2-chlorophenyl)amino)-4-oxo-4,5-dihydrofuran-3-carboxylate (13)

To a solution of 7-azaindole-3-carboxaldehyde (34, 0.74 g, 0.70 mmol) and ethyl 2-[(2-chlorophenyl)amino]-4-oxo-4,5-dihydrofuran-3-carboxylate (1.35 g, 4.8 mmol) in ethanol (32 mL), piperidine (0.048 mL, 0.48 mmol) was added at ambient temperature. The mixture was heated at reflux for 1 d. The hot reaction mixture was filtered, washed with hot ethanol and hexane, successively, and then dried to afford 13 (1.29 g, 66%) as a pale yellow solid. ¹H NMR (400 MHz, DMSO-d⁶) δ 12.31 (br. s., 1H), 10.65 (s, 1H), 8.22 (dd, J=1.5, 4.8 Hz, 1H), 7.72-7.82 (m, 4H), 7.53-7.62 (m, 2H), 6.92 (s, 1H), 6.81 (dd, J=4.6, 7.9 Hz, 1H), 4.29 (q, J=7.1 Hz, 2H), 1.30 (t, J=7.0 Hz, 3H). ¹³C NMR (101 MHz, DMSO-d⁶) δ 176.23, 171.73, 163.94, 149.37, 144.17, 142.45, 133.24, 132.04, 130.75, 130.49, 130.12, 129.50, 128.79, 128.68, 118.08, 117.02, 107.04, 103.90, 88.44, 59.78, 40.41, 40.20, 39.99, 39.78, 39.57, 15.01. MS m/z 410 [M+H]⁺.

4.1.13 Ethyl

### 5-((1H-pyrrolo[2,3-b]pyridin-3-yl)methylene)-2-((3-chlorophenyl)amino)-4-oxo-4,5-dihydrofuran-3-carboxylate (14)

Compound 14 was synthesized from 7-azaindole-3-carboxaldehyde (34) and ethyl 2-[(3-chlorophenyl)amino]-4-oxo-4,5-dihydrofuran-3-carboxylate prepared by GP-II as a pale yellow solid (40 mg, 27%), utilizing similar reaction conditions as described in GP-I. ¹H NMR (400 MHz, DMSO-d⁶) δ 12.36 (br. s, 1H), 10.63 (s, 1H), 8.26 (dd, J=1.4, 4.6 Hz, 1H), 8.01 (d, J=7.3 Hz, 1H), 7.82 (d, J=2.8 Hz, 1H), 7.76 (s, 1H), 7.50-7.61 (m, 3H), 6.89-6.96 (m, 2H), 4.28 (q, J=7.0 Hz, 2H), 1.30 (t, J=7.0 Hz, 3H). MS m/z 410 [M+H]⁺.

4.1.14 Ethyl

### 5-((1H-pyrrolo[2,3-b]pyridin-3-yl)methylene)-2-((4-chlorophenyl)amino)-4-oxo-4,5-dihydrofuran-3-carboxylate (15)

Compound 15 was synthesized from 7-azaindole-3-carboxaldehyde (34) and ethyl 2-[(4-chlorophenyl)amino]-4-oxo-4,5-dihydrofuran-3-carboxylate prepared by GP-II as a colorless solid (61 mg, 23%, purity 93%), utilizing similar reaction conditions as described in GP-I. ¹H NMR (400 MHz, DMSO-d⁶) δ 12.33 (br. s, 1H), 10.61 (s, 1H), 8.26 (d, J=3.3 Hz, 1H), 7.96 (d, J=7.5 Hz, 1H), 7.82 (d, J=2.8 Hz, 1H), 7.58-7.67 (m, 4H), 6.87-6.95 (m, 2H), 4.27 (q, J=7.0 Hz, 2H), 1.29 (t, J=7.0 Hz, 3H). MS m/z 410 [M+H]⁺.

4.1.15 Ethyl

### 5-((1H-pyrrolo[2,3-b]pyridin-3-yl)methylene)-2-((2-methoxyphenyl)amino)-4-oxo-4,5-dihydrofuran-3-carboxylate (16)

Compound 16 was synthesized from 7-azaindole-3-carboxaldehyde (34) and ethyl 2-[(2-methoxyphenyl)amino]-4-oxo-4,5-dihydrofuran-3-carboxylate prepared by GP-II as a colorless solid (304 mg, 74%), utilizing similar reaction conditions as described in GP-I. ¹H NMR (400 MHz, DMSO-d⁶) δ 12.34 (br. s., 1H), 10.35 (s, 1H), 8.25 (dd, J=1.4, 4.6 Hz, 1H), 8.02 (d, J=7.0 Hz, 1H), 7.85 (d, J=2.8 Hz, 1H), 7.66 (dd, J=1.4, 7.9 Hz, 1H), 7.41-7.51 (m, 1H), 7.28 (d, J=7.5 Hz, 1H), 7.12 (t, J=7.7 Hz, 1H), 6.88-6.99 (m, 2H), 4.28 (q, J=7.0 Hz, 2H), 3.85 (s, 3H), 1.30 (t, J=7.2 Hz, 3H). MS m/z 406 [M+H]⁺.

4.1.16 Ethyl

### 5-((1H-pyrrolo[2,3-b]pyridin-3-yl)methylene)-2-((3-methoxyphenyl)amino)-4-oxo-4,5-dihydrofuran-3-carboxylate (17)

Compound 17 was synthesized from 7-azaindole-3-carboxaldehyde (34) and ethyl 2-[(3-methoxyphenyl)amino]-4-oxo-4,5-dihydrofuran-3-carboxylate prepared by GP-II as a colorless solid (36 mg, 8.8%), utilizing similar reaction conditions as described in GP-I. ¹H NMR (400 MHz, DMSO-d⁶) δ 12.34 (br. s., 1H), 10.52 (s, 1H), 8.25 (dd, J=1.4, 4.6 Hz, 1H), 8.03 (d, J=7.0 Hz, 1H), 7.84 (d, J=2.8 Hz, 1H), 7.45 (t, J=8.0 Hz, 1H), 7.13-7.26 (m, 2H), 7.04 (dd, J=2.0, 8.3 Hz, 1H), 6.86-6.95 (m, 2H), 4.27 (q, J=7.0 Hz, 2H), 3.77 (s, 3H), 1.30 (t, J=7.15 Hz, 3H). MS m/z 406 [M+H]⁺.

4.1.17 Ethyl

### 5-((1H-pyrrolo[2,3-b]pyridin-3-yl)methylene)-2-((4-methoxyphenyl)amino)-4-oxo-4,5-dihydrofuran-3-carboxylate (18)

Compound 18 was synthesized from 7-azaindole-3-carboxaldehyde (34) and ethyl 2-[(4-methoxyphenyl)amino]-4-oxo-4,5-dihydrofuran-3-carboxylate prepared by GP-II as a colorless solid (92 mg, 22%), utilizing similar reaction conditions as described in GP-I. ¹H NMR (400 MHz, DMSO-d⁶) δ 12.31 (br. s., 1H), 10.47 (s, 1H), 8.24 (dd, J=1.5, 4.5 Hz, 1H), 7.94 (d, J=7.5 Hz, 1H), 7.77 (d, J=2.8 Hz, 1H), 7.50 (d, J=9.0 Hz, 2H), 7.09 (d, J=8.8 Hz, 2H), 6.81-6.92 (m, 2H), 4.26 (q, J=7.0 Hz, 2H), 3.87 (s, 3H), 1.29 (t, J=7.0 Hz, 3H). MS m/z 406 [M+H]⁺.

4.1.18 General Procedure of Construction of Furanone Ester by Method B (GP-III)

Ethyl 2-[(2-fluorophenyl)amino]-4-oxo-4,5-dihydrofuran-3-carboxylate. Diethyl malonate (1.0 mL, 6.6 mmol) was added dropwise to a solution of sodium hydride (60% w/w in oil, 317 mg, 7.9 mmol) in anhydrous tetrahydrofuran (12 mL) that was cooled with ice bath. The mixture was heated at reflux for 6 min. After cooling with ice bath, the reaction mixture was treated dropwise with chloroacetyl chloride (0.58 mL, 7.2 mmol) and stirred in the ice bath for 1 h, and then stirred at 45° C. for 1 h. The reaction mixture was cooled in the ice bath again, and then 2-fluoroaniline (0.76 mL, 7.9 mmol) was added dropwise. After stirring at ambient temperature for 17 h, the reaction mixture was heated at reflux for 2.5 h. The reaction mixture was allowed to cool to ambient temperature, diluted with aqueous saturated sodium bicarbonate solution, and extracted with ethyl acetate twice and with chloroform. The organic layer was washed with brine, dried over sodium sulfate and concentrated. The residue was triturated with ethanol-hexane to afford the title compound as a pale yellow solid (0.58 g, 33%). ¹H NMR (400 MHz, DMSO-d⁶) δ 10.31 (s, 1H), 7.58-7.64 (m, 1H), 7.32-7.42 (m, 2H), 7.24-7.30 (m, 1H), 4.69 (s, 2H), 4.23 (q, J=7.2 Hz, 2H), 1.26 (t, J=7.2 Hz, 3H). MS m/z 266 [M+H]⁺.

4.1.19 Ethyl

### 5-[(1H-pyrrolo[2,3-b]pyridin-3-yl)methylene]-2-[(2-fluorophenyl)amino]-4-oxo-4,5-dihydrofuran-3-carboxylate (19)

To a solution of 7-azaindole-3-carboxaldehyde (34, 50 mg, 0.34 mmol) and ethyl 2-[(2-fluorophenyl)amino]-4-oxo-4,5-dihydrofuran-3-carboxylate (0.10 g, 0.38 mmol) in ethanol (2 mL), piperidine (3.4 μL, 0.034 mmol) was added at ambient temperature. The mixture was heated at reflux for 1 d. The reaction mixture was filtered, washed with ethanol and hexane, successively, and then dried to afford 19 (37 mg, 27%) as an yellow solid. ¹H NMR (400 MHz, DMSO-d⁶) δ 12.31 (br. s, 1H), 10.55 (s, 1H), 8.22 (dd, J=1.4, 4.6 Hz, 1H), 7.84 (d, J=7.8 Hz, 1H), 7.67-7.77 (m, 2H), 7.55-7.63 (m, 1H), 7.45-7.53 (m, 1H), 7.37-7.44 (m, 1H), 6.92 (s, 1H), 6.83 (dd, J=4.6, 7.9 Hz, 1H), 4.28 (q, J=7.0 Hz, 2H), 1.30 (t, J=7.0 Hz, 3H). MS m/z 394 [M+H]⁺.

4.1.20 Ethyl

### 5-((1H-pyrrolo[2,3-b]pyridin-3-yl)methylene)-2-((4-fluorophenyl)amino)-4-oxo-4,5-dihydrofuran-3-carboxylate (20)

Compound 20 was synthesized from 7-azaindole-3-carboxaldehyde (34) and ethyl 2-[(4-fluorophenyl)amino]-4-oxo-4,5-dihydrofuran-3-carboxylate prepared by GP-III as a colorless solid (120 mg, 40%), utilizing similar reaction conditions as described in GP-I. ¹H NMR (400 MHz, DMSO-d⁶) δ 12.28 (br. s., 1H), 10.57 (br. s., 1H), 8.24 (br. s., 1H), 7.95 (d, J=7.8 Hz, 1H), 7.75 (br. s., 1H), 7.60-7.68 (m, 2H), 7.38 (t, J=8.3 Hz, 2H), 6.85-6.93 (m, 2H), 4.11-4.50 (m, 2H), 1.29 (t, J=6.9 Hz, 3H). MS m/z 394 [M+H]⁺.

4.1.21 Ethyl

### 5-((1H-pyrrolo[2,3-b]pyridin-3-yl)methylene)-4-oxo-2-(o-tolylamino)-4,5-dihydrofuran-3-carboxylate (21)

Compound 21 was synthesized from 7-azaindole-3-carboxaldehyde (34) and ethyl 2-(o-tolylamino)-4-oxo-4,5-dihydrofuran-3-carboxylate prepared by GP-III as a pale yellow solid (19 mg, 14%), utilizing similar reaction conditions as described in GP-I. 1 H NMR (400 MHz, DMSO-d⁶) δ 12.26 (br. s, 1H), 10.43 (s, 1H), 8.18-8.22 (m, 1H), 7.69-7.75 (m, 2H), 7.54 (d, J=7.8 Hz, 1H), 7.45-7.49 (m, 2H), 7.37-7.44 (m, 1H), 6.85 (s, 1H), 6.78 (dd, J=4.6, 7.9 Hz, 1H), 4.28 (q, J=7.2 Hz, 2H), 2.28 (s, 3H), 1.30 (t, J=7.0 Hz, 3H). MS m/z 390 [M+H]⁺.

4.1.22 Ethyl

### 5-((1H-pyrrolo[2,3-b]pyridin-3-yl)methylene)-4-oxo-2-(p-tolylamino)-4,5-dihydrofuran-3-carboxylate (22)

Compound 22 was synthesized from 7-azaindole-3-carboxaldehyde (34) and ethyl 2-(p-tolylamino)-4-oxo-4,5-dihydrofuran-3-carboxylate prepared by GP-III as a colorless solid (160 mg, 43%), utilizing similar reaction conditions as described in GP-I. ¹H NMR (400 MHz, DMSO-d⁶) δ 12.30 (br. s., 1H), 10.51 (br. s., 1H), 8.24 (d, J=3.4 Hz, 1H), 7.97 (d, J=7.3 Hz, 1H), 7.80 (br. s., 1H), 7.46 (d, J=7.8 Hz, 2H), 7.34 (d, J=8.3 Hz, 2H), 6.74-7.02 (m, 2H), 4.14-4.37 (m, 2H), 2.44 (s, 3H), 1.29 (t, J=6.9 Hz, 3H). MS m/z 390 [M+H]⁺.

4.1.23 Ethyl

### 5-((1H-pyrrolo[2,3-b]pyridin-3-yl)methylene)-2-((2,4-difluorophenyl)amino)-4-oxo-4,5-dihydrofuran-3-carboxylate (23)

Compound 23 was synthesized from 7-azaindole-3-carboxaldehyde (34) and ethyl 2-((2,4-difluorophenyl)amino)-4-oxo-4,5-dihydrofuran-3-carboxylate prepared by GP-II as an yellow solid (144 mg, 69%), utilizing similar reaction conditions as described in GP-I. ¹H NMR (400 MHz, DMSO-d⁶) δ 12.27 (br. s, 1H), 10.51 (s, 1H), 8.24 (dd, J=1.4, 4.6 Hz, 1H), 7.84 (d, J=7.8 Hz, 1H), 7.66-7.77 (m, 2H), 7.50-7.59 (m, 1H), 7.29 (t, J=7.8 Hz, 1H), 6.80-6.89 (m, 2H), 4.26 (q, J=7.0 Hz, 2H), 1.29 (t, J=7.0 Hz, 3H). MS m/z 412 [M+H]⁺.

4.1.24 Ethyl

### 5-((1H-pyrrolo[2,3-b]pyridin-3-yl)methylene)-2-((2,4-dimethylphenyl)amino)-4-oxo-4,5-dihydrofuran-3-carboxylate (24)

Compound 24 was synthesized from 7-azaindole-3-carboxaldehyde (34) and ethyl 2-((2,4-dimethylphenyl)amino)-4-oxo-4,5-dihydrofuran-3-carboxylate prepared by GP-III as a colorless solid (80 mg, 27%, purity 92%), utilizing similar reaction conditions as described in GP-I. ¹H NMR (400 MHz, DMSO-d⁶) δ 12.26 (br. s., 1H), 10.35 (br. s., 1H), 8.21 (d, J=3.9 Hz, 1H), 7.65-7.80 (m, 2H), 7.39 (d, J=8.3 Hz, 1H), 7.27 (s, 1H), 7.20 (d, J=7.8 Hz, 1H), 6.84 (s, 1H), 6.73 (dd, J=4.4, 7.8 Hz, 1H), 4.27 (q, J=7.0 Hz, 2H), 2.44 (s, 3H), 2.23 (s, 3H), 1.30 (t, J=6.9 Hz, 3H). MS m/z 404 [M+H]⁺.

4.1.25 Ethyl

### 5-((1H-pyrrolo[2,3-b]pyridin-3-yl)methylene)-4-oxo-2-(pyridin-2-ylamino)-4,5-dihydrofuran-3-carboxylate (25)

Compound 25 was synthesized from 7-azaindole-3-carboxaldehyde (34) and ethyl 2-(pyridin-2-ylamino)-4-oxo-4,5-dihydrofuran-3-carboxylate prepared by GP-III as a colorless solid (3.0 mg, 2.4%), utilizing similar reaction conditions as described in GP-I. ¹H NMR (400 MHz, DMSO-d⁶) δ 12.41 (br. s., 1H), 10.74 (br. s, 1H), 8.60 (d, J=3.3 Hz, 1H), 8.35 (d, J=7.8 Hz, 1H), 8.30 (dd, J=1.4, 4.6 Hz, 1H), 8.24 (br. s., 1H), 7.97 (dt, J=1.9, 7.8 Hz, 1H), 7.61 (d, J=8.0 Hz, 1H), 7.36-7.45 (m, 1H), 7.02-7.14 (m, 2H), 4.27 (q, J=7.0 Hz, 2H), 1.29 (t, J=7.0 Hz, 3H). MS m/z 377 [M+H]⁺.

4.1.26 Ethyl

### 5-((1H-pyrrolo[2,3-b]pyridin-3-yl)methylene)-4-oxo-2-(pyridin-3-ylamino)-4,5-dihydrofuran-3-carboxylate (26)

Compound 26 was synthesized from 7-azaindole-3-carboxaldehyde (34) and ethyl 2-(pyridin-3-ylamino)-4-oxo-4,5-dihydrofuran-3-carboxylate prepared by GP-III as a colorless solid (95 mg, 47%), utilizing similar reaction conditions as described in GP-I. ¹H NMR (400 MHz, DMSO-d⁶) δ 12.33 (br. s., 1H), 10.72 (br. s., 1H), 8.82 (br. s., 1H), 8.65 (br. s., 1H), 8.24 (br. s., 1H), 7.90-8.11 (m, 2H), 7.73 (br. s., 1H), 7.54-7.62 (m, 1H), 6.87-6.97 (m, 2H), 4.28 (d, J=6.9 Hz, 2H), 1.30 (t, J=6.4 Hz, 3H). MS m/z 377 [M+H]⁺.

4.1.27 Ethyl

### 5-((1H-pyrrolo[2,3-b]pyridin-3-yl)methylene)-4-oxo-2-(pyridin-4-ylamino)-4,5-dihydrofuran-3-carboxylate (27)

Compound 27 was synthesized from 7-azaindole-3-carboxaldehyde (34) and ethyl 2-(pyridin-4-ylamino)-4-oxo-4,5-dihydrofuran-3-carboxylate prepared by GP-III as an yellow solid (45 mg, 31%), utilizing similar reaction conditions as described in GP-I. H NMR (400 MHz, DMSO-d⁶) δ 12.30 (br. s., 1H), 11.18 (br. s, 1H), 8.59 (d, J=3.9 Hz, 2H), 8.28 (d, J=3.9 Hz, 1H), 8.19 (d, J=7.3 Hz, 1H), 7.88 (br. s., 1H), 7.55 (br. s., 2H), 7.02 (dd, J=4.4, 7.8 Hz, 1H), 6.93 (br. s., 1H), 4.23 (d, J=6.9 Hz, 2H), 1.26 (t, J=6.9 Hz, 3H). MS m/z 377 [M+H]⁺.

4.1.28 Ethyl

### 5-((1H-pyrrolo[2,3-b]pyridin-3-yl)methylene)-4-oxo-2-(pyrimidin-5-ylamino)-4,5-dihydrofuran-3-carboxylate (28)

Compound 28 was synthesized from 7-azaindole-3-carboxaldehyde (34) and ethyl 2-(pyrimidin-5-ylamino)-4-oxo-4,5-dihydrofuran-3-carboxylate prepared by GP-III as an yellow solid (13 mg, 10%), utilizing similar reaction conditions as described in GP-I. ¹H NMR (400 MHz, DMSO-d⁶) δ 12.34 (br. s., 1H), 10.80 (br. s., 1H), 9.19 (s, 1H), 9.02-9.12 (m, 2H), 8.25 (d, J=4.4 Hz, 1H), 8.03 (d, J=7.8 Hz, 1H), 7.71 (br. s., 1H), 6.86-7.00 (m, 2H), 4.28 (q, J=6.9 Hz, 2H), 1.30 (t, J=6.0 Hz, 3H). MS m/z 378 [M+H]⁺.

4.1.29 Ethyl

### 2-((1H-pyrazol-3-yl)amino)-5-((1H-pyrrolo[2,3-b]pyridin-3-yl)methylene)-4-oxo-4,5-dihydrofuran-3-carboxylate (29)

Compound 29 was synthesized from 7-azaindole-3-carboxaldehyde (34) and ethyl 2-((1H-pyrazol-3-yl)amino)-4-oxo-4,5-dihydrofuran-3-carboxylate prepared by GP-III as an yellow solid (200 mg, 35%), utilizing similar reaction conditions as described in GP-I. ¹H NMR (400 MHz, DMSO-d⁶) δ 13.07 (br. s., 1H), 12.34 (br. s., 1H), 10.46 (br. s., 1H), 8.27 (d, J=3.9 Hz, 1H), 8.14 (d, J=7.8 Hz, 1H), 7.91-8.20 (m, 2H), 7.08 (dd, J=4.7, 7.6 Hz, 1H), 6.94 (s, 1H), 6.47 (br. s., 1H), 4.26 (q, J=6.9 Hz, 2H), 1.29 (t, J=7.1 Hz, 3H). MS m/z 366 [M+H]⁺.

4.1.30 Ethyl

### 5-((1H-pyrrolo[2,3-b]pyridin-3-yl)methylene)-4-oxo-2-(quinolin-6-ylamino)-4,5-dihydrofuran-3-carboxylate (30)

Compound 30 was synthesized from 7-azaindole-3-carboxaldehyde (34) and ethyl 2-(quinolin-6-ylamino)-4-oxo-4,5-dihydrofuran-3-carboxylate prepared by GP-III as a pale yellow solid (140 mg, 32%), utilizing similar reaction conditions as described in GP-I. ¹H NMR (400 MHz, DMSO-d⁶) δ 12.29 (br. s., 1H), 10.82 (s, 1H), 9.01 (d, J=3.4 Hz, 1H), 8.39 (d, J=8.3 Hz, 1H), 8.23 (br. s., 1H), 8.17 (d, J=8.8 Hz, 1H), 8.11 (d, J=3.9 Hz, 1H), 8.00 (d, J=8.8 Hz, 1H), 7.88 (d, J=7.3 Hz, 1H), 7.83 (br. s., 1H), 7.62 (dd, J=4.2, 8.1 Hz, 1H), 6.94 (s, 1H), 6.26-6.51 (m, 1H), 4.30 (q, J=6.9 Hz, 2H), 1.32 (t, J=6.9 Hz, 3H). MS m/z 427 [M+H]⁺.

4.1.31 Ethyl

### 2-((1H-indazol-6-yl)amino)-5-((1H-pyrrolo[2,3-b]pyridin-3-yl)methylene)-4-oxo-4,5-dihydrofuran-3-carboxylate (31)

Compound 31 was synthesized from 7-azaindole-3-carboxaldehyde (34) and ethyl 2-((1H-indazol-6-yl)amino)-4-oxo-4,5-dihydrofuran-3-carboxylate prepared by GP-III as an yellow solid (40 mg, 10%, purity 93%), utilizing similar reaction conditions as described in GP-I. ¹H NMR (400 MHz, DMSO-d⁶) δ 13.22 (br. s., 1H), 12.24 (br. s., 1H), 10.66 (br. s., 1H), 8.22 (br. s., 1H), 8.12 (br. s., 1H), 7.91 (d, J=8.3 Hz, 1H), 7.71-7.86 (m, 3H), 7.33 (d, J=7.8 Hz, 1H), 6.89 (s, 1H), 6.33 (br. s., 1H), 4.29 (d, J=6.9 Hz, 2H), 1.31 (t, J=6.6 Hz, 3H). MS m/z 416 [M+H]⁺.

4.1.32 Ethyl

### 5-((1H-pyrrolo[2,3-b]pyridin-3-yl)methylene)-2-(benzylamino)-4-oxo-4,5-dihydrofuran-3-carboxylate hydrochloride (32)

7-Azaindole-3-carboxaldehyde (34) and ethyl 2-(benzylamino)-4-oxo-4,5-dihydrofuran-3-carboxylate prepared by GP-II were suspended in 2 N hydrochloric acid in ethanol (1 mL) and heated at reflux for 4 h. After cooling to ambient temperature, the liberated solid was collected by filtration, washed with ethanol and then isopropyl ether, and dried under reduced pressure to afford compound 32 (24 mg, 56%) as an yellow solid. ¹H NMR (400 MHz, DMSO-d⁶) δ 12.25-12.56 (m, 1H), 9.54 (br. s., 1H), 8.25-8.42 (m, 2H), 7.86 (s, 1H), 7.35-7.48 (m, 4H), 7.24-7.33 (m, 1H), 7.08-7.18 (m, 1H), 6.87 (s, 1H), 4.86 (d, J=6.5 Hz, 2H), 4.23 (q, J=7.2 Hz, 2H), 1.27 (t, J=7.0 Hz, 3H). Hydrochloride proton is missing. MS m/z 390 [M+H]⁺.

4.1.33 Ethyl

### 5-((1H-pyrrolo[2,3-b]pyridin-3-yl)methylene)-4-oxo-2-(phenethylamino)-4,5-dihydrofuran-3-carboxylate hydrochloride (33)

7-Azaindole-3-carboxaldehyde (34) and ethyl 2-(phenethylamino)-4-oxo-4,5-dihydrofuran-3-carboxylate prepared by GP-III were suspended in 2 N hydrochloric acid in ethanol (1 mL) and heated at reflux for 3 h. After cooling to ambient temperature, the liberated solid was collected by filtration, washed with ethanol and then isopropyl ether, and dried under reduced pressure to afford compound 33 (23 mg, 32%) as an yellow solid. ¹H NMR (400 MHz, DMSO-d⁶) δ 12.45 (br. s., 1H), 9.02 (t, J=6.2 Hz, 1H), 8.40 (d, J=6.8 Hz, 1H), 8.33 (dd, J=1.4, 4.6 Hz, 1H), 7.94 (d, J=2.3 Hz, 1H), 7.23-7.33 (m, 4H), 7.11-7.22 (m, 2H), 6.84 (s, 1H), 4.21 (q, J=7.2 Hz, 2H), 3.85 (q, J=6.9 Hz, 2H), 3.00 (t, J=7.3 Hz, 2H), 1.25 (t, J=7.0 Hz, 3H). Hydrochloride proton is missing. MS m/z 404 [M+H]⁺.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### CITATION LIST

### Patent Literature

PTL 1: U.S. Pat. No. 4,107,288.

PTL 2: U.S. Pat. No. 5,145,684.

1. A compound according to formula (I): 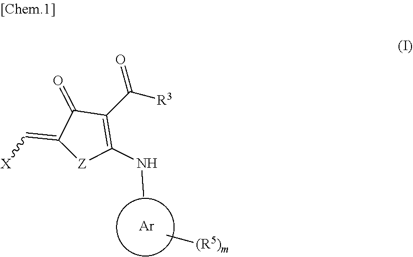
wherein
X is 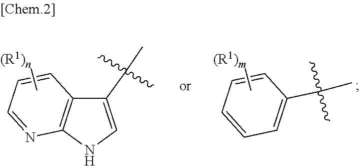
Z is O or S;
n is from 1-3;
m is from 0-4;
each R¹ and R⁵ are independently selected from the group consisting of:
halogen, hydrogen, hydroxy, C₁-C₈ alkyl, substituted C₁-C₈ alkyl, C₁-C₈ alkoxy, and substituted C₁-C₈ alkoxy;
R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded through a nitrogen atom to the carboxy group to form 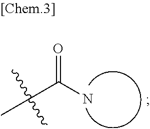 [SMILES: *C(N)=O]
Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
or a tautomer and/or a pharmaceutically acceptable salt thereof.

2. A compound according to formula (II): 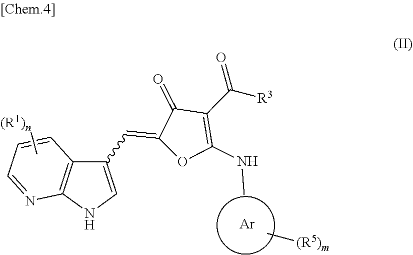
wherein
n is from 2-3;
m is from 0-4;
each R¹ and R⁵ are independently selected from the group consisting of:
halogen, hydrogen, hydroxy, C₁-C₈ alkyl, substituted C₁-C₈ alkyl, C₁-C₈ alkoxy, and substituted C₁-C₈ alkoxy;
R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded thru a nitrogen atom to the carboxy group to form 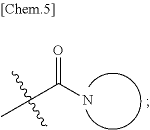 [SMILES: *C(N)=O]
Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
provided that at least two of the R¹ are not hydrogen;
or a tautomer and/or a pharmaceutically acceptable salt thereof.

3. The compound of claim 2, wherein Ar is

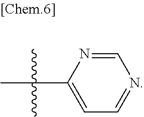 [SMILES: *c1ccncn1]

4. The compound of claim 2, wherein n is 2, m is 4, and Ar is Ph.

5. The compound of claim 2, wherein n is 3, m is 1, one R¹ is F substituted at the 4-position, one R¹ is F substituted at the 5-position, one R¹ is F substituted at the 6-position, R³ is 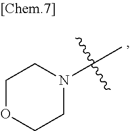 [SMILES: *N1CCOCC1]
Ar is 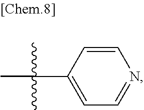 [SMILES: *c1ccncc1]
and R⁵ is Cl substituted at the 2-position.

6. The compound of claim 2, wherein n is 2, m is 2, R¹ is F substituted at the 5-position, R³ is 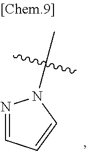 [SMILES: *n1cccn1]
Ar is Ph, one R⁵ is Cl substituted at the 2-position and one R⁵ is Cl substituted at the 4-position.

7. The compound of claim 2, wherein n is 2, m is 2, one R¹ is F substituted at the 4-position, one R¹ is F substituted at the 5-position, R³ is OMe, Ar is Ph, one R⁵ is OMe substituted at the 2-position and one R⁵ is OMe substituted at the 4-position.

8. The compound of claim 2, wherein n is 3, m is 1, one R¹ is F substituted at the 4-position, one R¹ is F substituted at the 5-position, one R¹ is F substituted at the 6-position, R³ is OEt, Ar is Ph, and R⁵ is F substituted at the 2-position.

9. The compound of claim 2, wherein n is 2, m is 0, one R¹ is OMe substituted at the 4-position, one R¹ is OMe substituted at the 5-position, R³ is hydroxy, and Ar is

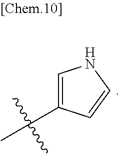 [SMILES: *c1cc[nH]c1]

10. The compound of claim 2, wherein n is 3, m is 1, one R¹ is Me substituted at the 4-position, one R¹ is Me substituted at the 5-position, one R¹ is Me substituted at the 6-position, R³ is hydroxy, Ar is Ph, and R⁵ is Cl substituted at the 2-position.

11. The compound of claim 2, wherein n is 2, m is 0, one R¹ is F substituted at the 4-position, one R¹ is F substituted at the 5-position, R³ is 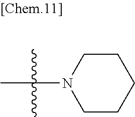 [SMILES: *N1CCCCC1]
and Ar is

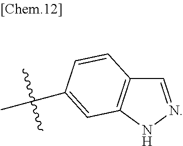 [SMILES: *c1ccc2cn[nH]c2c1]

12. The compound of claim 2, wherein n is 2, R³ is 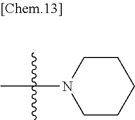 [SMILES: *N1CCCCC1]
and Ar is

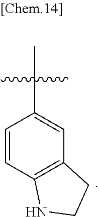 [SMILES: *c1ccc2c(c1)CCN2]

13. The compound of claim 2, wherein n is 2, R³ is 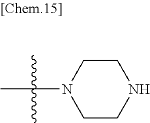 [SMILES: *N1CCNCC1]
and Ar is

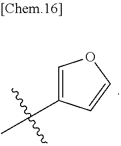 [SMILES: *c1ccoc1]

14. The compound of claim 2, wherein n is 2, R³ is 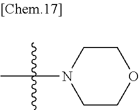 [SMILES: *N1CCOCC1]
and Ar is

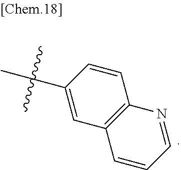 [SMILES: *c1ccc2ncccc2c1]

15. A compound according to formula (III): 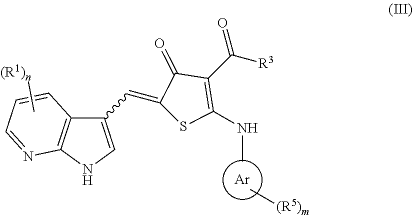
wherein
n is from 1-3;
m is from 0-4;
each R¹ and R⁵ are independently selected from the group consisting of:
halogen, hydrogen, hydroxy, C₁-C₈ alkyl, substituted C₁-C₈ alkyl, C₁-C₈ alkoxy, and substituted C₁-C₈ alkoxy;
R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded thru a nitrogen atom to the carboxy group to form 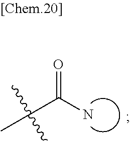 [SMILES: *C(N)=O]
Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
or a tautomer and/or a pharmaceutically acceptable salt thereof.

16. A compound according to formula (III): 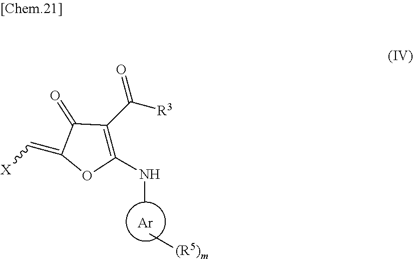
wherein
X is 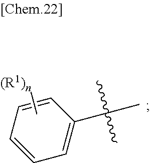
n is from 0-4;
m is from 0-4;
each R¹ and R⁵ are independently selected from the group consisting of:
halogen, hydrogen, hydroxy, C₁-C₈ alkyl, substituted C₁-C₈ alkyl, C₁-C₈ alkoxy, and substituted C₁-C₈ alkoxy;
R³ is selected from the group consisting of: hydroxy, C₁-C₈ alkoxy, substituted C₁-C₈ alkoxy, amino, C₁-C₈ substituted amino, C₃-C₁₂ heterocycle, and substituted C₃-C₁₂ heterocycle, such that the C₃-C₁₂ heterocycle is bonded through a nitrogen atom to the carboxy group to form 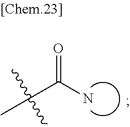 [SMILES: *C(N)=O]
Ar is an aromatic C₃-C₁₂ monocyclic or bicyclic group where each cyclic ring contains from zero up to three heteroatoms that are selected from the group consisting of: O, N, and S;
and wherein two R¹ and/or two R⁵ groups may join together to form a fused bicyclic ring system with the aromatic ring to which they are attached;
or a tautomer and/or a pharmaceutically acceptable salt thereof.

17. The compound of any one of the claims above, wherein the geometry of the exocyclic double bond is the Z-isomer.

18. A pharmaceutical composition comprising one or more compounds of any one of the claims above and one or more pharmaceutically acceptable excipients.

19. A kit including a composition comprising one or more compounds of any one of the claims above and instructions for use.

20. A method of selectively inhibiting Cdc7, the method comprising contacting one or more compounds of any one of the claims above with Cdc7 and at least one more enzyme selected from the group consisting of: CLK1, CLK2, GSK3α, GSK3β, DYRK1B, Erk1, Erk2, PIM1, and p70S6K.

21. The method of claim 20, wherein the inhibition is in the presence of 1 mM adenosine triphosphate (ATP).

22. The method of claim 20, wherein the selectivity for Cdc7 inhibition to inhibition of the at least one other enzyme is at least 8-fold.

23. The method of claim 20, wherein the compound inhibits Cdc7 with at least an IC₅₀ value of equal to or less than 1 μM.

24. The method of any of claims 20-23, wherein the contacting takes place in a cell.

25. A method of inhibiting Cdc7 comprising contacting a cell with one or more compounds of any one of the claims above.

26. The method of claim 25, wherein the cell is a Colo-205 cancer cell.

27. The method of claim 25, wherein the cell is a LS174T cancer cell.

28. The method of claim 25, wherein the cell is a DoHH2 cancer cell.

29. The method of claim 25, wherein the cell is a HeLa cancer cell.

30. A method for treating a cancer that is associated with Cdc7 overexpression as compared to Cdc7 expression in a non-cancerous control cell, the method comprising administering an effective amount of a composition comprising one or more compounds of any one of the claims above to a subject in need thereof.

31. The method of claim 30, wherein the cancer is selected from the group consisting of colon cancer, blood cancer, and cervical cancer.

32. The method of claim 30, wherein the cancer is colon cancer.

33. The method of claim 30, wherein the subject is a mammal.

34. The method of claim 30, wherein the subject is a human.

35. The use of one or more compounds of any one of the claims above for the manufacture of a medicament for treating cancer that is associated with Cdc7 overexpression as compared to Cdc7 expression in a non-cancerous control cell.
